(19) United States
(12) Patent Application Publication
(10) Pub. No.: US 2014/0349360 A1
(43) Pub. Date: Nov. 27, 2014

(54) METHODS AND SYSTEM FOR LIQUEFACTION, HYDROLYSIS AND FERMENTATION OF AGRICULTURAL FEEDSTOCKS

(71) Applicants: ENERGY BEET DESIGNS, LLC, Fresno, CA (US); THE REGENTS OF THE UNIVERSITY OF CALIFORNIA, Oakland, CA (US)

(72) Inventors: Ruihong ZHANG, Davis, CA (US); Steven M. ZICARI, Davis, CA (US); John E. DIENER, Five Points, CA (US); James R. TISCHER, Woodland, CA (US); Jeffrey H. MANTERNACH, Lake Oswego, OR (US); William C. PUCHEU, Tranquillity, CA (US); Jimmy L. MOORE, Fort Collins, CO (US); Joseph W. WINCKLER, Powhattan, KS (US)

(21) Appl. No.: 14/220,033

(22) Filed: Mar. 19, 2014

Related U.S. Application Data
(60) Provisional application No. 61/803,405, filed on Mar. 19, 2013.

(51) Int. Cl. C12P 7/14; C10L 1/02

(52) U.S. Cl. CPC C12P 7/14; C10L 1/02
USPC 435/162

(57) ABSTRACT

Treatment of agricultural biomass without separation of the biomass to extract fermentable feedstock, instead using a hydrolytic process upstream of the fermentation process, provides an efficient and cost-effective process for forming ethanol from agricultural biomass.

### CROSS-REFERENCE TO RELATED APPLICATIONS

This application claims priority to U.S. Provisional Patent Application No. 61/803,405 filed Mar. 19, 2013, the disclosure of which is incorporated herein by reference in its entirety for all purposes.

### FIELD OF THE INVENTION

The present invention relates generally to fermentation processes and systems for utilizing such processes. More specifically, the invention relates to fermentation processes and systems capable of processing substrates into useful target products, e.g., ethanol.

### BACKGROUND OF THE INVENTION

Industrial ethanol production is generally based on either ethylene conversion of fossil fuels such as oil and coal, or fermentation of carbohydrate-containing materials, such as agricultural products. Industrial fermentation of agricultural products for the production of target products, such as ethanol, is generally accomplished through batch processing.

Various economic and environmental factors have increased the demand for ethanol, and have created a complementary desire to reduce the use of fossil fuels. Environmental and economic factors drive a desire to decrease the quantity of discarded agricultural byproducts. The food industry disposes of significant quantities of fermentable material every year for lack of an efficient low cost means of fermenting its effluent. In particular, small-scale producers of fermentable byproducts lack an efficient means for converting the byproducts into useful target products, such as ethanol.

The production of ethanol for fuel applications is becoming increasingly important in the world. Ethanol is currently produced from the fermentation of cornstarch. In the United States, ethanol is currently primarily produced from the fermentation of cornstarch. Title IX of the 2002 Farm Bill and current USA Department of Energy and USA Department of Agriculture efforts are targeted at producing inexpensive ethanol from biomass resources. The goal is to promote a cleaner environment and reduce dependence on imported petroleum products.

It is known to use a variety of different types of feedstock to produce ethanol. It is also known to utilize a number of different methods for processing feedstock into ethanol. However, each of the different conventional methods suffers from one or more disadvantages, regardless of the type of feedstock used to produce ethanol. For example, conventional methods for producing ethanol require raw, unprocessed feedstock to be transported from the site where the feedstock is produced or stored to a remote processing plant. Transportation of raw, unprocessed feedstock from the site of the feedstock producer to the ethanol producing plant results in substantial equipment, labor, fuel, maintenance and repair costs. More particularly, the transportation of raw, unprocessed feedstock results in an ethanol yield (by weight) of approximately 33% of the feedstock (by weight). In addition, the transportation of raw, unprocessed feedstock results in byproduct at the ethanol producing plant which amounts to approximately 33% (by weight) of the feedstock (by weight). Additional transportation costs, including labor, fuel, maintenance and repair, are incurred in connection with the removal of the byproducts from the ethanol producing plant. Further, conventional methods for producing ethanol require large storage capacities at either or both the site of the feedstock producer and the ethanol producing plant.

The major challenges in converting lignocellulosic biomass to ethanol include high cost of dedicated biomass feedstock, pretreatment of lignocellulosic feedstock to release sugars for fermentation, poor fermentation of pentose sugars to ethanol by wild type microorganisms, and toxicity of biomass hydrolysates to both recombinant and wild type fermentative microorganisms.

Although sugar beets are a highly attractive feedstock for fermentation to ethanol, current methods for beet sugar refining for ethanol production are complex and energy intensive. A common process consists of cutting roots into fine strands, extracting sucrose with hot water, clarifying the juice with lime-carbonate addition, evaporating to low moisture content to produce concentrated sugar juice before it is used for fermentation. Because of these energy and water intensive processing requirements, a 2006 United States Department of Agriculture (USDA) report indicated that production costs for ethanol from sugar beet sugar and processing intermediates might be expected to be twice that as compared with con. However, increased biomass yield coupled with the elimination of several traditional sugar refining steps such as extraction, clarification, and crystallization can make commercial production attractive.

What is needed is a more economical and efficient method for producing ethanol from feedstocks with high concentrations of soluble saccharides, high pectin and low lignin content, such as sugar beets. The present invention provides such processes.

### BRIEF SUMMARY OF THE INVENTION

It has now been discovered that subjecting an agricultural feedstock to heat treatment prior to its introduction to an enzymatic hydrolysis mixture, significantly reduces the time required to reduce the viscosity of the hydrolysis mixture and, in some embodiments of the present invention, the time required to hydrolyze the feedstock sufficiently for the efficient production of fermentation products (e.g., ethanol) from the hydrolyzed feedstock.

In various embodiments, the present invention provides a new design for efficient production of ethanol from sugar beets and other fermentable feedstocks. In an exemplary embodiment, the invention provides a process for producing ethanol from the first agricultural feedstock that comprises significant amounts of soluble saccharides and has high pectin and low lignin contents and about 20% total solids (TS). Examplary feedstocks include sugar beets and melons. Soluble saccharides are precursors for ethanol production. The invention also provides a process for producing ethanol from remaining materials from the first ethanol fermentation process and other lignocellulosic materials, such as leaves, grasses and straw. The two processes can be integrated in a system. The method includes: (a) mechanically processing the first agricultural feedstock to prepare a first hydrolysis feedstock. The feedstock comprises a solid feedstock comprising said fermentable ethanol precursor; (b) in a hydrolysis vessel, contacting the first hydrolysis feedstock with a hydrolytic enzyme capable of liquefying the solid feedstock under conditions sufficient to liquefy the solid feedstock, forming a first fermentation substrate; and (c) in a fermentation vessel, contacting the first fermentation substrate with a microorganism capable of converting the fermentation substrate to ethanol under conditions sufficient to convert the fermentable ethanol precursor to a first ethanol fraction.

Other objects, advantages and aspects of the invention are provided in the detailed description below.

### BRIEF DESCRIPTION OF THE DRAWINGS

FIG. 1A-1C shows reducing sugars (RSS) yield (g/g TS) as an effect of different enzyme loading in sugar beet leaf

FIG. 2 shows ethanol production from sugar beets during SSF (initial TS content 10%).

FIG. 3 shows ethanol production sugar beet SSF (initial TS content 20%).

FIG. 4 shows ethanol production of sugar beet SSF trials with different treatment conditions (initial TS content 20%).

FIG. 5A-5C shows fermentation product and sugar analyses for sugar beet SSF at several time points with different treatment conditions (initial TS content 20%).

FIG. 6 shows continuous stirred tank reactor sugar beet fermentation apparatus.

FIG. 7 shows continuous stirred tank reactor sugar beet fermentation apparatus.

FIG. 8 shows ethanol production in the 5th 2-kg sugar beet fermentation trial.

FIG. 9 shows sugars and products for the 2-kg sugar beet fermentation trials A-D.

FIG. 10 shows 2nd stage ethanol fermentation using E. coli KO11 from the 1^{st} 2-kg sugar beet trial stillage.

FIG. 11 shows 2nd stage ethanol fermentation using S. cerevisea with the 3^{rd} 2-kg sugar beet trial stillage.

FIG. 12-FIG. 17 describe the operational configurations of an exemplary system and functional aspects of the various unit operations.

FIG. 18 shows the beneficial impact on liquefaction of adding yeast for a single enzyme loading condition.

FIG. 19 shows results from the pilot trial for average apparent viscosity.

FIG. 20 shows results from enzyme trials for average major soluble component concentrations.

FIG. 21 shows individual results of ethanol concentrations for each enzyme trial.

FIG. 22 shows individual results of average fermentation byproducts for each enzyme trial.

FIG. 23 shows individual results of average solubilized unconsumed carbohydrates.

FIG. 24 illustrates an exemplary pilot plant process block flow diagram.

FIG. 25A-25B illustrates an exemplary pilot plant process flow/piping and instrumentation diagram (part 1). FIG. 25A illustrates an enlarged view of section A. FIG. 25B illustrates an enlarged view of section B.

FIG. 26A-26B illustrates an exemplary pilot plant process flow/piping and instrumentation diagram (part 2). FIG. 26A illustrates an enlarged view of section A. FIG. 26B illustrates an enlarged view of section B.

FIG. 27 is a plot showing the viscosity of hydrolysis mixtures of beet feedstock pre-treated with heat and with no such pretreatment. The curves correspond to various enzymatic conditions. The viscosity of the hydrolysis mixtures was measured at approximately 20% total solids (TS). The asterisk refers to measurement as done with an Anton-Parr ST-59 Building Material Cell Stirrer at a shear rate of 50 sec⁻¹ and 50° C. The legend refers to the loading of Novozymes CTEC2 cellulase:HTEC2 hemicellulase:NS22119 Pectinase in units of FPU/g-TS:XU/g-TS:PGU/g-TS, respectively, where TS refers to “total solids”.

### DETAILED DESCRIPTION OF THE INVENTION

### I. Introduction

The current invention provides a novel method for converting various feedstocks to ethanol. In various embodiments, the feedstock is an agricultural feedstock. The method of the invention is a simple, efficient and cost-effective process for converting fermentable feedstock into ethanol.

In various embodiments, the invention provides processes for producing ethanol from sugar beets without a cooking or extraction step (e.g., heating in liquid) to separate fermentable saccharides from the solid fraction of the feedstock.

Sugar beets are unique biofuel feedstocks as having high soluble sugar and low lignin contents, coupled with high pectin and hemicellulosic cell wall fractions with a high capacity to entrain liquids, causing initial rheological phenomena to be dominated by wet particle interactions. This feedstock is being examined as a biofuel feedstock in the US and several other countries. Beets are extremely efficient at producing easily fermentable sugars and can provide approximately twice the ethanol production yield as starch-based corn ethanol per area.

Current methods for beet sugar refining for ethanol production consists of cutting roots into fine strands, extracting sucrose with hot water, clarifying the juice with lime-carbonate addition, evaporating to low moisture content to produce concentrated sugar juice before it is used for fermentation. Because of these energy and water intensive processing requirements, a 2006 United States Department of Agriculture (USDA) report indicated that production costs for ethanol from sugar beet sugar and processing intermediates might be expected to be twice that as compared with con. However, increased biomass yield coupled with the elimination of several traditional sugar refining steps such as extraction, clarification, and crystallization can make commercial production attractive. The present invention provides methods eliminating one or more of these undesirable steps.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

### II. Definitions

The following terms are used in the claims of the patent as filed and are intended to have their broadest meaning consistent with the requirements of law. Where alternative meanings are possible, the broadest meaning is intended. All words used in the claims are used in the normal, customary usage of grammar and the English language.

“Contaminating microorganisms” means reactive microorganisms that do not participate in a useful manner, or that participate in a harmful manner, with the production of a target product from a substrate.

“Continuous flow” means a fermentation process in which target product is output from the system while most of the mash remains in one or more reaction vessels, and in which emptying of reaction vessels is generally not required to maintain production of the target product. “Continuous flow” includes fermentation systems in which the fermentation microorganism cell mass in a reaction vessel is maintained at a viable level while the target product is removed from the system.

“Fermentation microorganisms” means reactive microorganisms involved in a microbial-controlled production of a target product from an organic or inorganic substrate.

“Mash” means the contents of a reaction vessel, which may include: feed substrate, nutrients, fermentation microorganisms, water, minerals, the target product, and miscellaneous metabolic by-products in small quantities.

“Reactive microorganisms” means microorganisms that react with a substrate, including both fermentation microorganisms and contaminating microorganisms.

“Sterilized” and “sterilization” means the reduction or destruction of contaminating microorganisms naturally present in the feedtock. An exemplary sterilization reduces the population of contaminating microorganisms to a level sufficiently low as to cause no significant impediment to the fermentation of the feedstock to produce ethanol.

“Feedstock” means a material capable of being at least partially converted into a target product by fermentation microorganisms. The feedstock can include a first feedstock and a second feedstock. Exemplary feedstocks include beets, apple pomace, peach pomace, banana skin, apricot peel, mango peel, citrus peel, orange peel, grapefruit peel, lemon peel, lime peel; potato pulp, tomato pulp, pumpkin pulp, carrot pulp, avocado fruit or pomace. In an exemplary embodiment, the first feedstock is not a lignocellulosic feedstock, e.g., straw, corn stovers, or grasses.

“Beets” in terms of this application include all plants of the species Beta vulgaris. These include, for example, beetroot, sugar beet and fodder beet as well as chard.

The term “cellulolytic activity” is defined herein as a biological activity that hydrolyzes a cellulose-containing material. Cellulolytic protein may hydrolyze microcrystalline celluose or other cellulosic substances, thereby decreasing the mass of insoluble cellulose and increasing the amount of soluble sugars. The reaction can be measured by the detection of reducing sugars with p-hydroxybenzoic acid hydrazide, a high-performance-liquid-chromatography (HPLC), or an electrochemical sugar detector. Determination of cellulase activity, measured in terms of Filter Paper Units (FPU) quantifies the amount of catalytic activity present in a sample by measuring the dilution of enzyme required to release 2.0 mg of reducing sugar equivalents from filter paper in 1 h at 50° C. and pH 4.8.

“Soluble saccharides” refers to saccharides having significant solubility in water. Exemplary soluble saccharides include sucrose, glucose, fructose and/or a combination thereof “Soluble saccharides” can be produced in the process of the method by hydrolysis of oligo- and poly-saccharides.

### III. The Embodiments

### A. The Method

In various embodiments, the present invention provides a new design for production of ethanol from various fermentable feedstocks. In an exemplary embodiment, the feedstock is an agricultural feedstock. Thus, in an exemplary embodiment, the invention provides a process for preparing ethanol from a first agricultural feedstock comprising a significant fraction of a soluble saccharide, which is a fermentable ethanol precursor, and about 20% total solids (TS). The method includes: (a) mechanically processing the first agricultural feedstock to prepare a first hydrolysis feedstock, and heating the feedstock to sterilize/pasteurize it. The feedstock comprises a solid feedstock comprising said fermentable ethanol precursor; (b) in a hydrolysis vessel, contacting the first hydrolysis feedstock with a hydrolytic enzyme capable of liquefying the solid feedstock under conditions sufficient to liquefy the solid feedstock, forming a first fermentation substrate; and (c) in a fermentation vessel, contacting the first fermentation substrate with a microorganism capable of converting the fermentation substrate to ethanol under conditions sufficient to convert the fermentable ethanol precursor to a first ethanol fraction.

In an exemplary embodiment, the process of the invention is a continuous flow fermentation process. In various embodiments, the hydrolysis step, the fermentation step or both are facilitated by the cellulolytic activity of one or more enzymes and/or reactive microorganisms.

In an exemplary embodiment, the feedstock is sugar beet root.

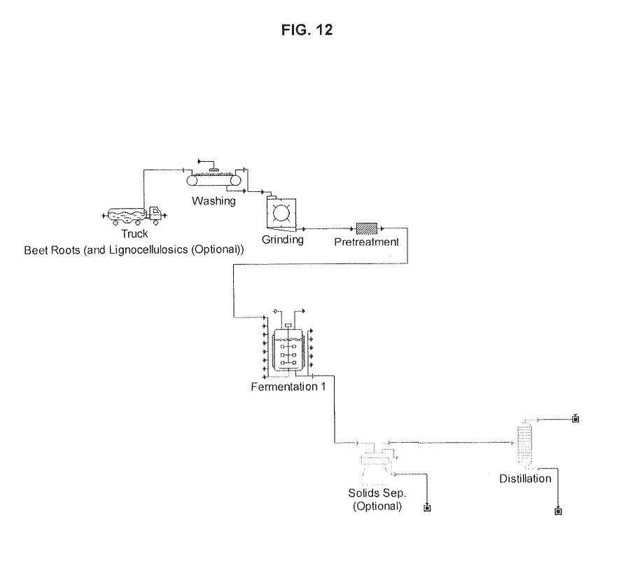

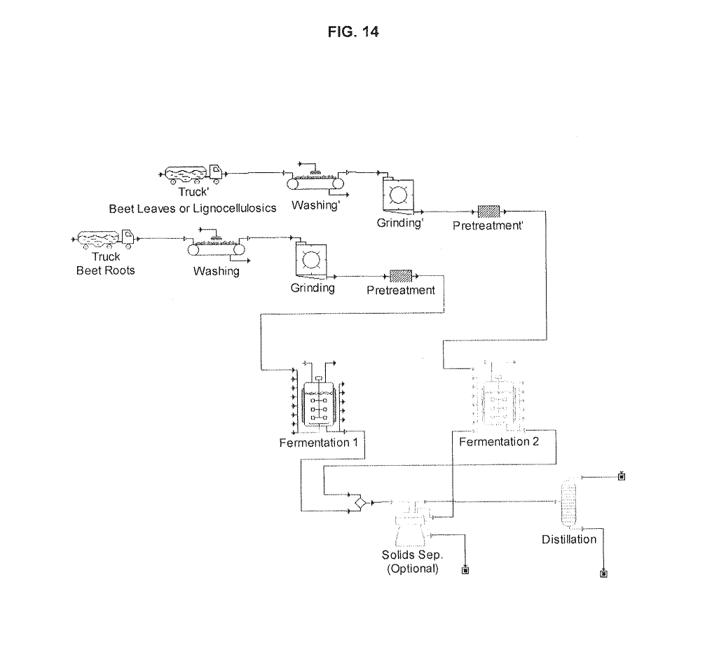

As exemplified in FIG. 12, the feedstock (e.g., beet roots (and additional agricultural feedstocks)) is processed to remove dirt and other detritus, and then it is processed to reduce its size (e.g., by grinding). The feedstock is pretreated (e.g., by hydrolysis; FIG. 14) and hence passed into the fermentation vessel where it is retained in contact with a fermentative microorganism and/or enzyme. The product of fermentation (e.g., ethanol) is removed by distillation and the solids are optionally separated.

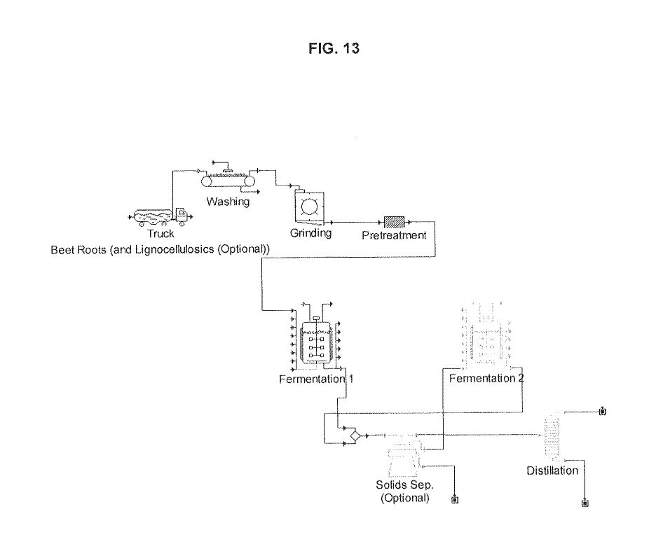

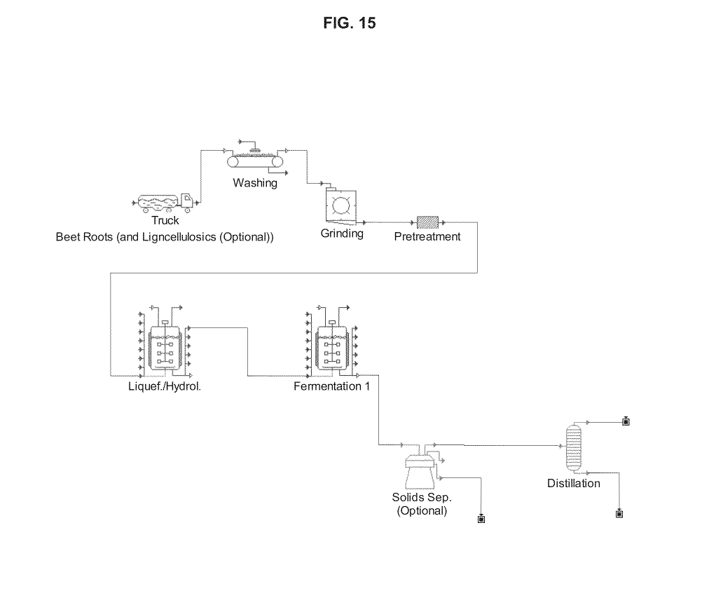

The process of the invention can include more than one fermentation step and the device can include more than one fermentation vessel. As illustrated in FIG. 13 and FIG. 15, the product of a first fermentation can be separated from solids and passed through into a second fermentation vessel. The product from the second fermentation can be combined with the product from the first fermentation and the mixed products distilled. As will be apparent to the skilled artisan, in various embodiments, the hydrolysis vessel and the fermentation vessel can be the same vessel or different vessels. The process and device of the invention can utilize one or more hydrolysis vessels (e.g., 2, 3, 4, 5 or more) and/or one or more fermentation vessels (e.g., 2, 3, 4, 5 or more).

The process can be conducted at ambient temperature or, in some embodiments, under higher temperatures. For example it is within the scope of the invention to augment the process by, prior to (b), heating the first hydrolysis feedstock. In an exemplary embodiment the contents of the vessel are heated to a temperature of from about 70° C. to about 130° C. for a time of from about 5 minutes to about 120 minutes (e.g., from about 95° C. to about 100° C. for a time of about 15 minutes to about 20 minutes). In various embodiments, the contents of the vessel are heated to about 70° C. for a time of about 120 minutes.

In an exemplary embodiment, the heating does not lead to any significant separation (e.g., extraction) of fermentable feedstock from the biomass, and its purpose is rather to decrease microbial activity, particularly the activity of contaminating microorganisms in the hydrolysis feedstock (i.e., sterilize). In various embodiments, the amount of additional fermentable feedstock separated (e.g., extracted) from the hydrolysis feedstock by the heating is not more than about 2%, not more than about 5%, not more than about 7% or not more than about 10% of the total fermentable feedstock in the biomass. In this regard, the heating is not an extractive “cooking” or separation step.

In various embodiments, the heating step is not accompanied by the addition of a significant amount of water to the hydrolysis feedstock (e.g., not more than about 2%, not more than about 5%, not more than about 7%, or not more than about 10% on a w/w basis of water:hydrolysis feedstock.

In an exemplary embodiment, the heating is accomplished by exposing the feedstock to steam. In various embodiments, the heating is accomplished by exposing the feedstock to steam under pressure. In various embodiments, the heating is performed at a temperature of from about 70° C. to about 130° C. The duration of the heating is of a length sufficient to reduce the microbial population of the feedstock. In various embodiments, the microbial population of the feedstock is reduced sufficiently that the ethanol yield derived from fermentation of a hydrolysate of this feedstock is at least about 10%, at least about 15%, at least about 20%, at least about 25% or at least about 25% greater than the ethanol yield from a sample identical except it has not been heat treated. In an exemplary embodiment, in which the feedstock is heated, fermentation of the corresponding hydrolysate produces ethanol in an amount of at least about 0.3 g EtOH/g initial dry solids, at least about 0.35 g EtOH/g initial dry solids, at least about 0.4 g EtOH/g initial dry solids. In contrast, an identical method using feedstock that is not heat treated produces less than about 0.3 g EtOH/g initial dry solids, or less than about 0.25 g ETOH/g initial dry solids. In various embodiments, this yield of ethanol produced after the brief hydrolysis period described hereinbelow.

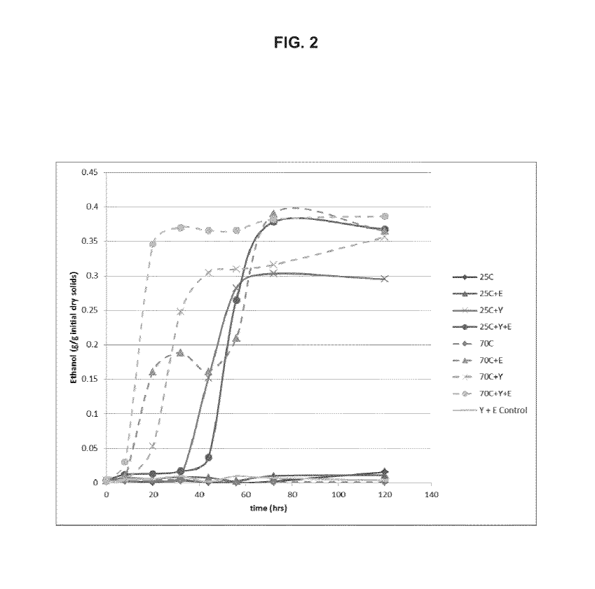

In various embodiments, the process of the invention, utilizing feedstock processed through an initial heat sterilization/pasteurization step, produces significant EtOH in less than about 30 hours, less than about 25 hours, less than about 20 hours or less than about 15 hours. In an exemplary embodiment, the amount of EtOH produced is at least about 0.1 g EtOH/g initial dry solids, at least about 0.2 g EtOH/g initial dry solids, at least about 0.3 g EtOH/g initial dry solids, at least about 0.35 g EtOH/g initial dry solids, or at least about 0.40 g EtOH/g initial dry solids at any of the enumerated time points. See, e.g., FIG. 2.

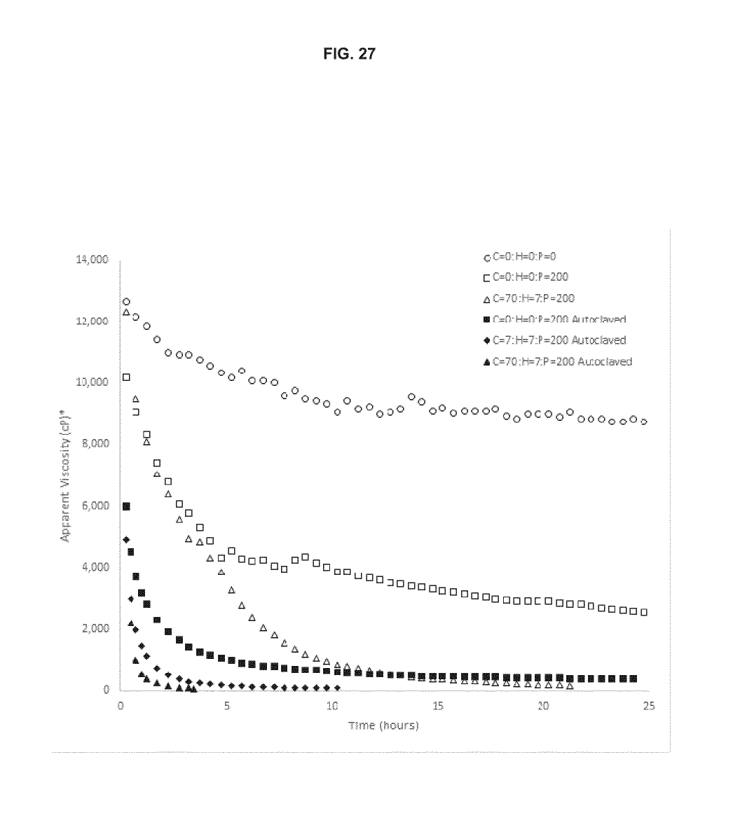

In various embodiments, heat pretreatment of the feedstock also significantly reduces the time required for liquefaction in the hydrolysis phase (FIG. 27). In FIG. 27, the decrease in viscosity with time of six samples of beet feedstock under various treatment conditions is compared. The curves with open symbols were not pretreated with heat before being submitted to enzymatic hydrolysis, those marked by solid symbols were heat pretreated. Each curve was measured at a TS content of the mixture of about 20%. Note that the viscosity of the samples not heat pretreated drops below ˜1,000 cp only after more than 10 hours of incubation with high enzyme loading. In contrast, feedstock that is heat pretreated, e.g., autoclaved (121° C., 20 minutes) is liquefied to a viscosity below this value in only about 1-4 hours. In an exemplary embodiment, the viscosity of the hydrolysis mixture of heat pretreated feedstock is reduced to under 1,000 cp in under four hours by treatment with pectinase alone, hemicellulase alone or cellulase alone. In certain embodiments, the viscosity of the hydrolysis mixture of heat pretreated feedstock is reduced to under 1,000 cp in under four hours by treatment with pectinase alone and one or more of hemicellulase and cellulase.

In various embodiments, the feedstock is heat pretreated as set forth above and is submitted to enzymatic hydrolysis for not more than one about hour, not more than about two hours, not more than about three hours, not more than about four hours, not more than about five hours, or not more than eight hours. An exemplary hydrolysis mixture has, at the end of the hydrolysis treatment, a viscosity of less than about 1000 cp.

Thus, exemplary benefits of thermal pretreatment include 1) reduction in microbial contamination; and 2) pretreatment effect for faster enzymatic liquefaction. This benefit may be attributable to solubilization/loosening of some cellular and structural biomass components as well as potential destruction of enzyme inhibitors sometimes present in live plant cells such as pectin methylesterase inhibitors (PMEI).

In various embodiments, the process of the invention includes mechanically or chemically treating the feedstock prior to its introduction into the hyrolysis vessel. In some cases, pretreatment methods of processing begin with a physical preparation of the biomass, e.g., size reduction of raw biomass feedstock materials, such as by cutting, grinding, crushing, smashing, shearing or chopping. In some embodiments, methods (e.g., mechanical methods) are used to reduce the size and/or dimensions of individual pieces of biomass. In some cases, loose feedstock (e.g., recycled paper or switchgrass) is pretreated by shearing or shredding. Screens and/or magnets can be used to remove oversized or undesirable objects such as, for example, rocks or nails from the feed stream.

Feed pretreatment systems can be configured to produce feed streams with specific characteristics such as, for example, specific maximum sizes, specific length-to-width, or specific surface areas ratios. As a part of feed pretreatment, the bulk density of feedstocks can be controlled (e.g., increased).

In some embodiments, the biomass is in the form of a fibrous material that includes fibers provided by shearing the biomass. For example, the shearing can be performed with a rotary knife cutter.

The temperature of the apparatus and process can be maintained at any useful level. In an exemplary embodiment, the hydrolysis vessel is maintained at a temperature of from about 25° C. to about 90° C., e.g., from about 35° C. to about 80° C., e.g., from about 40° C. to about 75° C.

The pretreatment can be performed in a batch or continuous flow type process.

In an exemplary embodiment, the hydrolysis reaction liquefies the feedstock. In various embodiments, the liquefying the feedstock reduces the mechanical strength of the first agricultural feedstock by at least about 50%, at least about 60%, at least about 70%, at least about 80%, at least about 90%, or about 100%. In an exemplary embodiment, the alteration in mechanical strength is measured by determining a change in viscosity of the hydrolysis mixture. Methods of determining viscosity are known in the art and are appropriate for or adaptable to the method of the invention.

The viscosity can be determined at any stage of the hydrolysis reaction and it can be determined as many times as thought desirable. In various embodiments, process decisions are informed by the viscosity of the hydrolysis feedstock. In an exemplary embodiment, the viscosity of the first hydrolysis feedstock in the hydrolysis vessel is quantified, and when the viscosity has reached a predetermined viscosity threshold, said first hydrolysis feedstock is transferred to the fermentation vessel. In an exemplary embodiment the predetermined viscosity threshold is from about 500 cp to about 1000 cp. An exemplary method of measuring the viscosity of the hydrolysis feedstock used the Anton Paar building material cell at 25° C. and a shear rate of 50-s.

### B. Feedstock

Generally, any biomass material that is or includes carbohydrates composed of one or more saccharide units or that include one or more saccharide units is a feedstock that can be processed by any of the methods described herein. As used herein, biomass includes, cellulosic, lignocellulosic, hemicellulosic, starch, and lignin-containing materials. For example, the biomass material can be cellulosic or lignocellulosic materials, or starchy materials, such as kernels of corn, grains of rice or other foods, or materials that are or that include one or more low molecular weight sugars, such as sucrose or cellobiose.

In various embodiments the primary fermentable substrate contains a soluble saccharide e.g., sucrose, glucose, fructose and/or a combination thereof.

Exemplary feedstocks include those with a high soluble carbohydrate, high pectin and low lignin content. In various embodiments, the feedstock is an agricultural feedstock. Exemplary agricultural feedstocks include roots, fruits and vegetables, including melons, potatoes and beets. When the feestock is a beet, it is generally preferred that it is a sugar beet.

Exemplary secondary feedstocks include paper, paper products, wood, wood-related materials, particle board, leaves, grasses, rice hulls, bagasse, cotton, jute, hemp, flax, bamboo, sisal, abaca, straw, corn cobs, rice hulls, coconut hair, algae, seaweed (e.g., giant seaweed), water hyacinth, cassava, coffee beans, coffee bean grounds (used coffee bean grounds), cotton, synthetic celluloses, or mixtures of any of these.

Fiber sources of use as second feedstocks include cellulosic fiber sources, including paper and paper products (e.g., polycoated paper and Kraft paper), and lignocellulosic fiber sources, including wood, and wood-related materials, e.g., particle board. Other suitable fiber sources include natural fiber sources, e.g., grasses, rice hulls, bagasse, cotton, jute, hemp, flax, bamboo, sisal, abaca, straw, corn cobs, rice hulls, coconut hair; fiber sources high in α-cellulose content, e.g., cotton; and synthetic fiber sources, e.g., extruded yarn (oriented yarn or un-oriented yarn). Natural or synthetic fiber sources can be obtained from virgin scrap textile materials, e.g., remnants or they can be post consumer waste, e.g., rags. When paper products are used as fiber sources, they can be virgin materials, e.g., scrap virgin materials, or they can be post-consumer waste. Aside from virgin raw materials, post-consumer, industrial (e.g., offal), and processing waste (e.g., effluent from paper processing) can also be used as fiber sources. Also, the fiber source can be obtained or derived from human (e.g., sewage), animal, or plant waste. Additional fiber sources have been described in the art, for example, see U.S. Pat. Nos. 6,448,307, 6,258,876, 6,207,729, 5,973,035 and 5,952,105.

Plant biomass and lignocellulosic biomass include organic matter (woody or non-woody) derived from plants, especially matter available on a sustainable basis. Examples include biomass from agricultural or food crops (e.g., sugarcane, sugar beets or corn kernels) or an extract therefrom (e.g., sugar from sugarcane and corn starch from corn), agricultural crop wastes and residues such as corn stover, wheat straw, rice straw, sugar cane bagasse, and the like. Plant biomass further includes, but is not limited to, trees, woody energy crops, wood wastes and residues such as softwood forest thinnings, barky wastes, sawdust, paper and pulp industry waste streams, wood fiber, and the like. Additionally grass crops, such as switchgrass and the like have potential to be produced on a large-scale as another plant biomass source. For urban areas, the plant biomass feedstock includes yard waste (e.g., grass clippings, leaves, tree clippings, and brush) and vegetable processing waste.

In some embodiments, secondary feedstock can include lignocellulosic feedstock can be plant biomass such as, but not limited to, non-woody plant biomass, cultivated crops, such as, but not limited to, grasses, for example, but not limited to, grasses, such as switchgrass, cord grass, rye grass, miscanthus, reed canary grass, or a combination thereof, or sugar processing residues such as bagasse, or beet pulp, agricultural residues, for example, soybean stover, corn stover, rice straw, rice hulls, barley straw, corn cobs, wheat straw, canola straw, rice straw, oat straw, oat hulls, corn fiber, recycled wood pulp fiber, sawdust, hardwood, for example aspen wood and sawdust, softwood, or a combination thereof. Further, the lignocellulosic feedstock can include cellulosic waste material such as, but not limited to, newsprint, cardboard, sawdust, and the like. Lignocellulosic feedstock can include one species of fiber or alternatively, lignocellulosic feedstock can include a mixture of fibers that originate from different lignocellulosic feedstocks. Furthermore, the lignocellulosic feedstock can comprise fresh lignocellulosic feedstock, partially dried lignocellulosic feedstock, fully dried lignocellulosic feedstock, or a combination thereof.

In an exemplary embodiment, the secondary feedstock is leaves from the plant from which the first feedstock is derived, e.g., beet leaves.

Microbial biomass includes biomass derived from naturally occurring or genetically modified unicellular organisms and/or multicellular organisms, e.g., organisms from the ocean, lakes, bodies of water, e.g., salt water or fresh water, or on land, and that contains a source of carbohydrate (e.g., cellulose). Microbial biomass can include, but is not limited to, for example protists (e.g., animal (e.g., protozoa such as flagellates, amoeboids, ciliates, and sporozoa) and plant (e.g., algae such alveolates, chlorarachniophytes, cryptomonads, euglenids, glaucophytes, haptophytes, red algae, stramenopiles, and viridaeplantae)), seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture systems.

In an exemplary embodiment, the process of the invention utilizes more than one feedstock. By way of illustration, in one embodiment, the process of the invention includes introducing into the process a second agricultural feedstock. The second feedstock can be any useful feedstock, such as those disclosed herein, however, in an exemplary embodiment, the second feedstock is an agricultural feedstock, e.g., a lignocellulosic feedstock. Exemplary lignocellulosic biomass feedstocks include leaves, grass, straw and a combination thereof. In one embodiment, the lignocellulosic biomass feedstock is sugar beet leaf biomass.

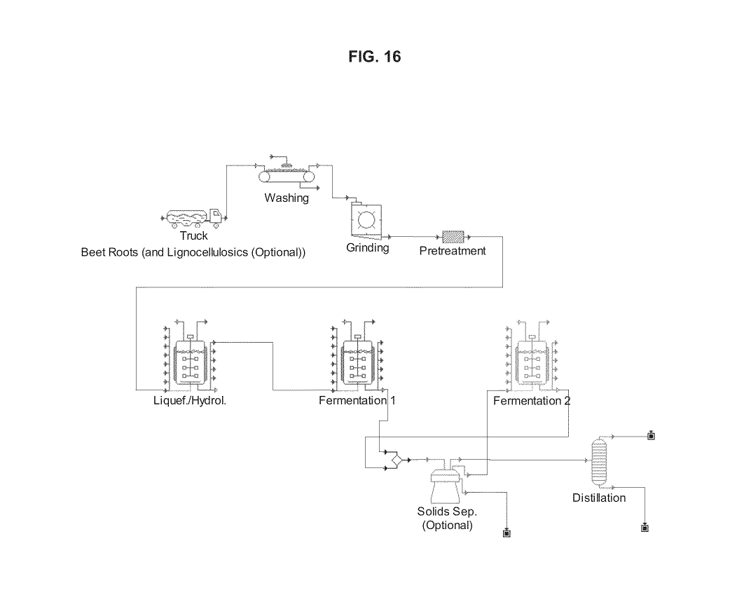

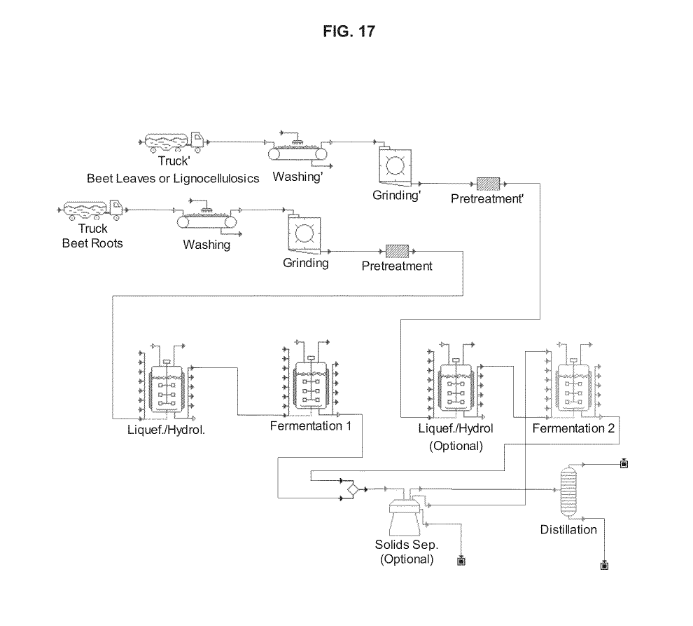

In various embodiments of the process of the invention in which the process further comprises the introduction of a second feedstock, an exemplary method further comprises one or more steps selected from: (f) mechanically processing the second feedstock (e.g., agricultural feedstock) to prepare a second hydrolysis feedstock comprising a solid feedstock and a liquid feedstock each comprising a second fermentable ethanol precursor; (g) in a second hydrolysis vessel, contacting the second hydrolysis feedstock with a hydrolytic enzyme capable of liquefying the solid feedstock under conditions sufficient to liquefy the solid feedstock, forming a second fermentation substrate; and (h) in a second fermentation vessel, contacting the second fermentation substrate with an organism (e.g., a yeast) expressing an enzyme capable of converting the second fermentation substrate to ethanol under conditions sufficient to convert the fermentable ethanol precursor to a second ethanol fraction (FIG. 16, FIG. 17).

As illustrated in FIG. 16, the second feedstock can be pretreated by submitting it to hyrdolysis (FIG. 17) or another process. The second feedstock can then be transferred to a fermentation vessel where it is contacted with a fermentative microorganism and/or enzyme for the production of the desired product of fermentation. As discussed herein, the product can be separated from solids and further purified by distillation. In various embodiments, the process does not include separating the solid feedstock and said liquid feedstock prior to (h).

In various embodiments, the process includes removing one or more portion of a reaction component of the hydrolysis or fermentation reaction. In one embodiment, the method further comprises: (d) removing at least a portion of the ethanol produced by the fermentation from the fermentation vessel, such that the fermentation vessel comprises stillage.

In various embodiments, the method of the invention further comprises: (e) contacting the stillage with an organism (e.g., a yeast) capable of converting the stillage to ethanol under conditions sufficient to convert stillage to a third ethanol fraction.

As will be appreciated by those of skill in the art, once ethanol is produced in the fermentation vessel, all or a fraction of the ethanol can be removed from the fermentation vessel. When ethanol is removed from the device and process of the invention, it can be removed by any useful method including, without limitation, distillation and membrane separation.

In an exemplary embodiment, after fermentation, the resulting fluids are distilled using, for example, a “beer column” to separate ethanol and other alcohols from the majority of water and residual solids. In various embodiments, the vapor exiting the beer column is about 35% by weight ethanol and fed to a rectification column. A mixture of nearly azeotropic (92.5%) ethanol and water from the rectification column can be purified to pure (99.5%) ethanol using vapor-phase molecular sieves. In an exemplary embodiment, the beer column bottoms are sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling point compounds.

Once removed, the ethanol can be advanced to the product stage or it can be reintroduced into the device and process of the invention. For example, ethanol removed from the fermentation vessel can be added to a member selected from the first fermentation vessel, the second fermentation vessel and a combination thereof.

Although the invention is exemplified by the production of ethanol, alcohols produced using the materials described herein can include ethanol but are not limited to this alcohol, and can include other monohydroxy alcohols or a polyhydroxy alcohol, e.g., ethylene glycol or glycerin. Examples of alcohols that can be produced include, but are not limited to, methanol, ethanol, propanol, isopropanol, butanol, e.g., n-, sec- or t-butanol, ethylene glycol, propylene glycol, 1,4-butane diol, glycerin or mixtures of these alcohols.

In various embodiments, lignin is produced as a byproduct of the process of the invention. Lignin is a phenolic polymer that is typically associated with cellulose in biomass, e.g., plants. In some instances the methods described herein will generate lignin that can be obtained (e.g., isolated or purified) from the biomass feedstock described herein. In some embodiments, the lignin obtained from any of the processes described herein can be, e.g., used as a plasticizer, an antioxidant, in a composite (e.g., a fiber resin composite), as a filler, as a reinforcing material, and in any of the pharmaceutical compositions described herein.

In addition, as described above, lignin-containing residues from primary and pretreatment processes has value as a high/medium energy fuel and can be used to generate power and steam for use in plant processes. However, such lignin residues are a new type of solid fuel and there may be little demand for it outside of the plant boundaries, and the costs of drying it for transportation may subtract from its potential value. In some cases, gasification of the lignin residues can be used to convert it to a higher-value product with lower cost.

### C. Enzymes and Microorganisms

The process of the invention can be practiced using one or more enzymes introduced into the hydrolysis and/or fermentation process as a reagent, or it can be practiced using one or more microorganisms expressing one or more enzymes useful in the hydrolysis and/or fermentation process.

When a microorganism is used, the microorganism can be a natural microorganism or an engineered microorganism. For example, the microorganism can be a bacterium, e.g., a cellulolytic bacterium, a fungus, e.g., a yeast, a plant or a protist, e.g., an algae, a protozoa or a fungus-like protist, e.g., a slime mold. Mixtures of organisms can be utilized.

Generally, various microorganisms can produce a number of useful products by operating on feedstock, e.g., fermenting treated biomass materials. For example, alcohols, organic acids, hydrocarbons, hydrogen, proteins or mixtures of any of these materials can be produced by hydrolysis, fermentation or other processes.

In an exemplary embodiment, the one or more enzyme hydrolyses components of the biomass selected from an oliogsaccharide, a polysaccharide and a combination thereof. To effect this transformation, in various embodiments the enzyme utilized is selected from a cellulase, a hemi-cellulase, a pectinase a β-glucosidase and a combination thereof. In an exemplary embodiment, the oligosaccharide is pectin and the enzyme of use in the process of the invention hydrolyses pectin. In various embodiments, the hydrolytic enzyme is a combination of one or more cellulase and one or more pectinase.

In some embodiments, materials that include cellulose are first treated with the enzyme, e.g., by combining the materials and the enzyme in an aqueous solution. This material can then be combined with the microorganism. In other embodiments, the materials that include the cellulose, the one or more enzymes and the microorganism are combined concurrently, e.g., by combining in an aqueous solution.

Also, to aid in the breakdown of the treated biomass materials, the treated biomass materials can be further treated e.g., with heat, a chemical (e.g., mineral acid, base or a strong oxidizer such as sodium hypochlorite), and/or an enzyme.

During fermentation, sugars released from cellulolytic hydrolysis or saccharification, are fermented to, e.g., ethanol, by a fermenting microorganism such as yeast. Suitable fermenting microorganisms have the ability to convert carbohydrates, such as glucose, fructose, xylose, arabinose, mannose, galactose, oligosaccharides, or polysaccharides into fermentation products. Fermenting microorganisms include strains of the genus Sacchromyces spp. e.g., Sacchromyces cerevisiae (baker's yeast), Saccharomyces distaticus, and Saccharomyces uvarum; the genus Kluyveromyces, e.g., species Kluyveromyces marxianus, and Kluyveromyces fragilis; the genus Candida, e.g., Candida pseudotropicalis, and Candida brassicae; the genus Clavispora, e.g., species Clavispora lusitaniae and Clavispora opuntiae; the genus Pachysolen, e.g., species Pachysolen tannophilus; the genus Bretannomyces, e.g., species Bretannomyces clausenii; the genus Pichia, e.g., species Pichia stipitis; and the genus Saccharophagus, e.g., species Saccharophagus degradans (Philippidis, 1996, “Cellulose Bioconversion Technology”, in Handbook on Bioethanol: Production and Utilization, Wyman, ed., Taylor & Francis, Washington, D.C., 179-212).

Commercially available yeast include, for example, Red Star™./Lesaffre Ethanol Red (available from Red Star/Lesaffre, USA); FALI™ (available from Fleischmann's Yeast, a division of Burns Philip Food Inc., USA); SUPERSTART™ (available from Alltech, now Lallemand); GERT STRAND™ (available from Gert Strand AB, Sweden); and FERMOL™ (available from DSM Specialties).

Bacteria that can ferment biomass to ethanol and other products include, e.g., Zymomonas mobilis and Clostridium thermocellum (Philippidis, 1996, supra). Leschine et al. (International Journal of Systematic and Evolutionary Microbiology 2002, 52, 1155-1160) describe an anaerobic, mesophilic, cellulolytic bacterium from forest soil, Clostridium phytofermentans sp. nov., which converts cellulose to ethanol.

Fermentation of biomass to ethanol and other products can be carried out using certain types of thermophilic or genetically engineered microorganisms, such as Thermoanaerobacter species, including T. mathranii, and yeast species such as Pichia species. An example of a strain of T. mathranii is A3M4 described in Sonne-Hansen et al. (Applied Microbiology and Biotechnology 1993, 38, 537-541) or Ahring et al. (Arch. Microbiol. 1997, 168, 114-119).

Yeast and Zymomonas bacteria can be used for fermentation or conversion. The optimum pH for yeast is from about pH 4 to 5, while the optimum pH for Zymomonas is from about pH 5 to 6. Typical fermentation times are about 24 to 96 hours with temperatures in the range of 26° C. to 40° C., however, thermophilic microorganisms may prefer higher temperatures.

Several additional factors can also be considered when selecting suitable microorganisms for use in the methods described herein. For example, if the microorganisms are to be used to generate a health product for use with animals or humans, or if the microorganisms are to be used as or in the production of a food, the microorganisms selected will typically be non-pathogenic and/or generally regarded as safe (GRAS). In addition, the microorganisms selected should be capable of producing large quantities of the desired product or should be able to be modified to produce large quantities of the desired product. In some embodiments, the microorganisms can also be commercially available and/or efficiently isolated, readily maintainable in culture, genetically stable and/or well characterized. Selected microorganisms can be wild type (e.g., unmodified) or genetically modified microorganisms (e.g., mutated organisms). In some embodiments, a genetically modified microorganism can be adapted to increase its production of the desired product and/or to increase the microorganisms tolerance to one or more environmental and/or experimental factors, for example, the microorganism can be modified (e.g., engineered) to tolerate temperature, pH, acids, bases, nitrogen, and oxygen levels beyond a range normally tolerated by the microorganism. Alternatively or in addition, the microorganisms can be modified (e.g., engineered) to tolerate the presence of additional microorganisms. In some embodiments, the microorganisms can be modified (e.g., engineered) to grow at a desired rate under desired conditions.

Enzymes that break down biomass, such as cellulose, to lower molecular weight carbohydrate-containing materials, such as glucose, are referred to as cellulolytic enzymes or cellulase; this process is referred to a “saccharification”. These enzymes can be a complex of enzymes that act synergistically to degrade crystalline cellulose. Examples of cellulolytic enzymes include: endoglucanases, cellobiohydrolases, and cellobiases (β-glucosidases). For example, cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble β-1,4-linked dimer of glucose. Finally cellobiase cleaves cellobiose to yield glucose.

A cellulase is capable of degrading biomass and can be of fungal or bacterial origin. Suitable enzymes include cellulases from the genera Bacillus, Pseudomonas, Humicola, Fusarium, Thielavia, Acremonium, Chrysosporium and Trichoderma, and include species of Humicola, Coprinus, Thielavia, Fusarium, Myceliophthora, Acremonium, Cephalosporium, Scytalidium, Penicillium or Aspergillus (see, e.g., hsEP 458162), especially those produced by a strain selected from the species Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes can also be obtained from Chrysosporium, preferably a strain of Chrysosporium lucknowense. Additionally, Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, e.g., EP 458162) can be used.

Cellulolytic enzymes produced using recombinant technology can also be used (see, e.g., WO 2007/071818 and WO 2006/110891).

The cellulolytic enzymes used can be produced by fermentation of the above-noted microbial strains on a nutrient medium containing suitable carbon and nitrogen sources and inorganic salts, using procedures known in the art (see, e.g., Bennettand LaSure (eds.), More Gene Manipulations in Fungi, Academic Press, CA 1991). Suitable media are available from commercial suppliers or can be prepared according to published compositions (e.g., in catalogues of the American Type Culture Collection). Temperature ranges and other conditions suitable for growth and cellulase production are known in the art (see, e.g., Bailey and 011 is, Biochemical Engineering Fundamentals, McGraw-Hill Book Company, NY, 1986).

Treatment of cellulose with cellulase is usually carried out at temperatures between 30° C. and 65° C. Exemplary cellulases of use in the invention are active over a range of pH of about 3 to 7. A saccharification step can last for any useful duration, e.g., up to about 120 hours. The cellulase enzyme dosage achieves a sufficiently high level of cellulose conversion. For example, an appropriate cellulase dosage is typically between about 5.0 and about 50 Filter Paper Units (FPU) per gram of cellulose. The FPU is a standard measurement and is defined and measured according to Ghose (1987, Pure and Appi. Chem. 59:257-268).

In particular embodiments, Cellic CTEC2 and HTEC2 (Novozymes), and P2611 Pectinase from Aspergillus aceleatus (Sigma-Aldrich) are utilized as the enzyme system at loadings of 0.7, 0.07, and 0.05 mL per 100 gram of substrate, respectively. These enzyme products are multiple enzyme cocktails with multiple activities, mainly exoglucanase, endoglucanase, hemicellulase, beta-glucosidase, pectin esterase, and polygalacturonase. The mixed cocktail has approximate activities of >100 FPU/ml, >5000 XU/ml, and >10000 PGU/ml. The pH optimums for the enzymes are all in the 5.0-5.5 range, with a temperature optimum around 50° C.

### The Process

The invention is further illustrated by reference to certain exemplary processes. For example FIG. 12 is a process diagram depicting an exemplary process for biorefining a biomass such as beet roots and/or other optional or additional materials such as lignocellulosic biomass. As shown in FIG. 12, after the biomass is transported to the processing site or equipment, the biomass is cleaned or washed to remove undesired materials such as soil. The cleaned biomass is then grinded to desired sizes and conveyed to a fermentation system (e.g., fermentor) or systems for fermentation. Once the fermentation is completed, distillation process is conducted to collect desired products such as ethanol. In various embodiments, the biorefining process includes other optional or additional processes. For example, in some embodiments, after the biomass being grinded but prior to fermentation, the biomass is undergone some pretreatments such as heating or “pre-steaming.” In some embodiments, separation of solids is conducted after fermentation and before distillation.

In various embodiments, the process for biorefining a biomass includes a plurality of fermenters or fermentation systems, that are disposed or connected in series, in parallel or in the combination of series and parallels. For instance, FIG. 13 illustrates a two stage fermentation that includes a second or secondary fermentation such as Fermentation 2. In the illustrated embodiment, the Fermentation 2 is conducted on the remaining biomass after the first fermentation, e.g., Fermentation 1.

In some embodiments, different fermentation systems have separate biomass supply lines (e.g., transportation, washing and grinding). For instance, FIG. 14 illustrates a process that includes a separate supply line for each of the two fermentation systems. Accordingly, the biomass being supplied to Fermentation 1 can be the same as or different than the biomass being supplied to Fermentation 2. As an example, FIG. 14 illustrates feeding beet roots to Fermentation 1 whereas feeding beet leaves and/or other optional materials such as lignocellulosics to Fermentation 2. In addition to having its own supply line, in some embodiments, Fermentation 2 is also conducted on the remaining biomass after Fermentation 1.

In various embodiments, the process for biorefining a biomass includes additional processes or steps, such as liquefaction and/or hydrolysis, to enhance the throughput of the biofuel and/or improve the overall performance. For instance, FIG. 14 depicts a process similar to FIG. 12 and further including a liquefaction and/or hydrolysis process conducted after grinding the biomass but prior to fermentation; FIG. 15 depicts a process similar to FIG. 13 and further including a liquefaction and/or hydrolysis process conducted after grinding the biomass but prior to the first fermentation, e.g., Fermentation 1. In some embodiments, the process further includes a plurality of liquefaction and/or hydrolysis processes. For instance, FIG. 16 depicts a process similar to FIG. 14 and further including two liquefaction and/or hydrolysis processes, one conducted prior to Fermentation 1 and the other prior to Fermentation 2.

In various embodiments, solid and liquid residuals produced by the process set forth herein, e.g., stillage remaining after removal of EtOH produced in the process, is transferred to an anaerobic digestion apparatus for conversion to methane and other gases. Exemplary biogasification apparati are known and are of use in the present invention. See, for example, Zhang et al., U.S. Pat. Nos. 7,556,737; 7,316,921; 7,015,028; and 6,342,378.

The following examples are provided to illustrate exemplary embodiments of the invention.

### EXAMPLES

### Example 1

### Enzymatic Pretreatment of Sugar Beet Leaves

### Objectives

Since sugar beet is an important crop in California and several other areas in the United States and sugar beet leaves account for 40-50% of total biomass wet weight, it can become a significant biomass feedstock for biofuel production with production as high as 35-44 tons of leaves per acre in California. This study was carried out to investigate enzymatic liquefaction and saccharification of the leaves for production of the fermentable sugar and other compounds that can be later used for biofuel production through fermentation. The results from this study are applicable for other herbaceous biomass materials.

### Methods

Sugar beet leaves from mature ENC115 variety were collected from the research farm at University of California-Davis. The fresh and dried leave samples were analyzed for moisture content (MC), total solid (TS), volatile solid (VS), and chemical compositions. Enzymatic liquefaction and saccharification of the leaves were investigated using a mix of cellulases (CTEC2), hemicellulases (HTEC2), from Novozymes and pectinases (P2611) from Sigma-Aldrich. Various enzyme loadings were investigated to determine desirable enzyme loadings as shown in Table 2. In each experiment, 1 g leave sample of 14% TS content were tested in 2-mL tubes, at 50° C., and mixed at a low speed of 20 rpm. The total hydrolysis time was 7 days. Samples were withdrawn daily to measure total reducing sugars (RSS), ethanol, sugar acids and galacturonic acid.

TABLE 2
Experimental Design for Enzymatic Hydrolysis of Sugar Beet Leaves

| | Enzyme Loadings | | |
|---|---|---|---|
| | | | P2611 |
| | CTEC | HTEC | (PGU/g |
| Treatment | (FPU/g cellulose) | (XU/g hemicellulose) | pectin) |
| 1 | 30, 60, 90 | 60 | 90 |
| 2 | 60 | 30, 60, 90 | 90 |
| 3 | 60 | 60 | 30, 60, 90 |

### Results and Discussion

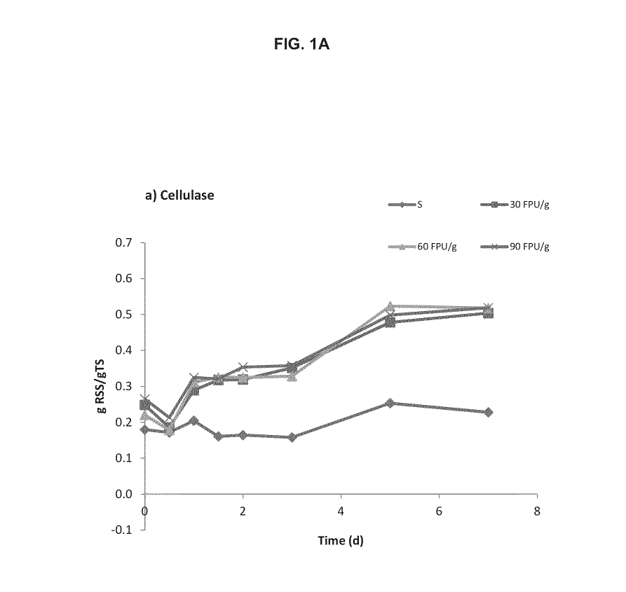

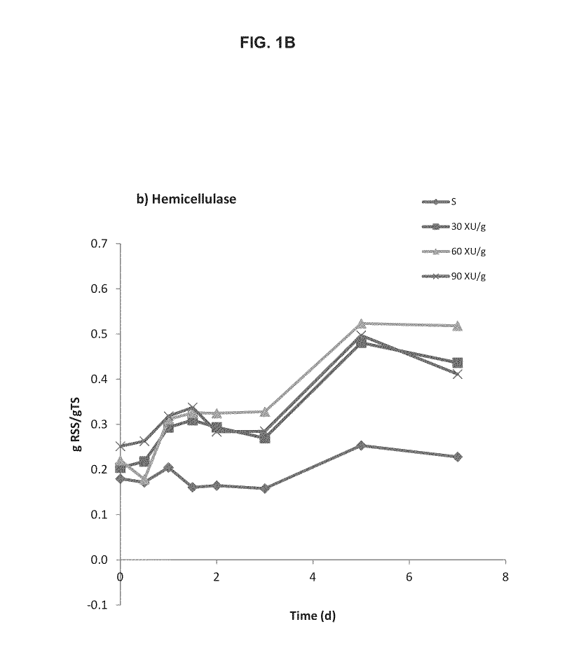

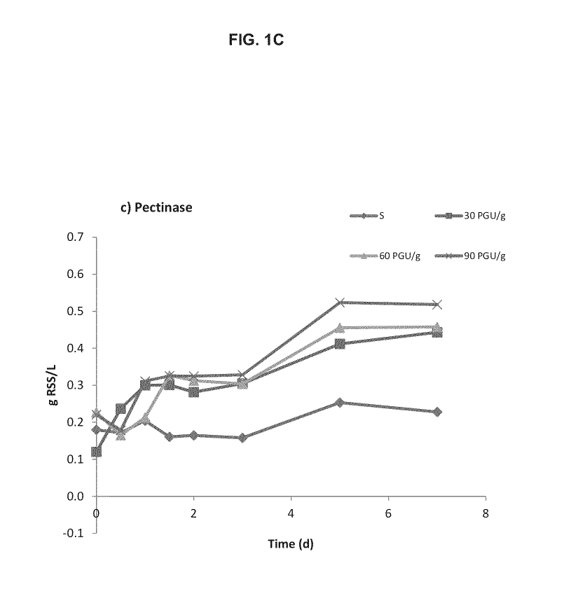

The beet leaves contained 15.1% TS which is composed of cellulose, hemicellulose and pectin (reported as uronic acid) as primarily structural carbohydrates (39.3% TS) accounting for 10.5%, 14.9% and 13.9% of TS respectively. Total non-structural carbohydrates make up 17.3% of TS. Maximum reducing sugar (RSS) concentration and yield reached 65 g/L and 0.52 g/g TS, respectively, after seven days of enzymatic saccharification at an enzyme mixture of 60 FPU cellulase/g cellulose, 60 XU hemicellulase/g hemicellulose, and 90 PGU pectinase/g pectin. Compared to the hydrolysis without enzyme addition, RSS concentration and yield are 31 g/L and 0.23 g/g TS. The addition of enzyme increases RSS concentration and yield of 110% and 126%, respectively. The maximum RSS yield accounts for 76% of the initial cellulose, hemicelluloses and pectin content in the sugar beet leaves. Increase of cellulase and pectinase concentrations improved RSS concentration and yield, thus enzymatic hydrolysis as shown in FIG. 1.

### Example 2

### Batch Bottle Hydrolysis and Fermentation of Sugar Beet Root

### Objectives

The objectives of initial studies were to evaluate the performance of enzymatic hydrolysis and anaerobic ethanol production using Saccharomyces cerevisea with sugar beets (SB) at initial total solids of ˜10% and ˜20% under non-sterile conditions. The raw SB has approximately 20% TS, however, dilution to approximately 10% TS with water allows initial mixing and material handling with most standard reactor and pump designs. No supplemental nutrients or pH control were provided. To evaluate whether microbial contamination could be reduced, allowing more carbon to be channeled to ethanol production, four pretreatment methods were also evaluated: autoclaving (115° C., 20 min), preheating (70° C., 2 h), adding antibiotics and initial pH reduction.

### Methods

Sugar beet roots (variety B4430R) were grown and harvested at the University of California, Davis, in July 2011. The beet roots were washed and ground using a 5 HP Hobart Cutter Mixer (Rough Grind), and frozen at −20° C. Upon thawing for use, beets were further food processed for 1 minute using a Cuisinart 11-cup Food Processor (Fine Grind) to ensure uniform consistency. Total solid content (TS) was measured by drying samples at 105° C. in an oven for 2 days and expressed as the weight ratio of dry to wet sample. The volatile solid content (VS) was determined using a muffle furnace at 550° C.

Sugarbeet was then placed into 250 mL media bottles using 100 g for a nominal 20% solids condition, and 50 g beet plus 50 g water for the nominal 10% solids conditions. Media bottles were outfitted with rubber stoppers and one-way valves to allow gas release only. Duplicate bottles for each condition were tested and control samples without yeast (Y), enzyme (E), or either were run simultaneously. Once loaded into the bottles, samples were subjected to various pretreatments as described below.

A first set of experiments were conducted to evaluate differences in ethanol production at 10% and 20% initial solids content and subjected to either a 70° C. pretreatment for 2-hours achieved by submersion of bottles in a 70° C. water bath, or holding at room temperature (25° C.) for the same amount of time. A second set of experiments were conducted to evaluate the effect of four pretreatment conditions, including a) autoclaving the SB at 115° C. for 20 min; b) adjusting initial pH of SB from 6.5 to 3.5 by sulfuric acid; c) preheating the SB at 70° C. for 2 h; d) addition of industrial antibiotics (5 ppm penicillin or 5 ppm erythromycin) to control Gram-positive bacteria. Heated samples were then cooled to 37° C. prior to addition of yeast and/or enzymes as described below.

Novozymes CTEC2 (Cellulase, 120 FPU/mL), Novozymes HTEC2 (Hemicellulase, 131 XU/mL), and Novozymes 188 (beta-glucosidase, 250 CBU/mL) were obtained from Novozyme Inc. (USA) and pectinase P2611 (3800 PGU/mL) was purchased from sigma chemical. The enzyme mixture was prepared by mixing these four enzymes with a volume ratio of 14:15:3.5:1 immediately prior to the fermentation. The enzyme loading is approximately 1.68 mL of enzyme mixture per 100 g of sugar beet root (wet basis). An enzyme blank with enzyme and yeast was performed at batch scale and approximately 2.8 g/L of ethanol are attributed to production from the enzymes and subtracted from final fermentation concentrations.

Industrial yeast (Bioferm XR, North American Bioproducts Corporation) cells aerobically propagated per vendor instructions using 5% yeast-peptone-dextrose (YPD) media and harvested by centrifuge for addition at a concentration of 0.5 mg cells/g dry solid. Anaerobic fermentation was carried out at 37° C. with orbital shaking.

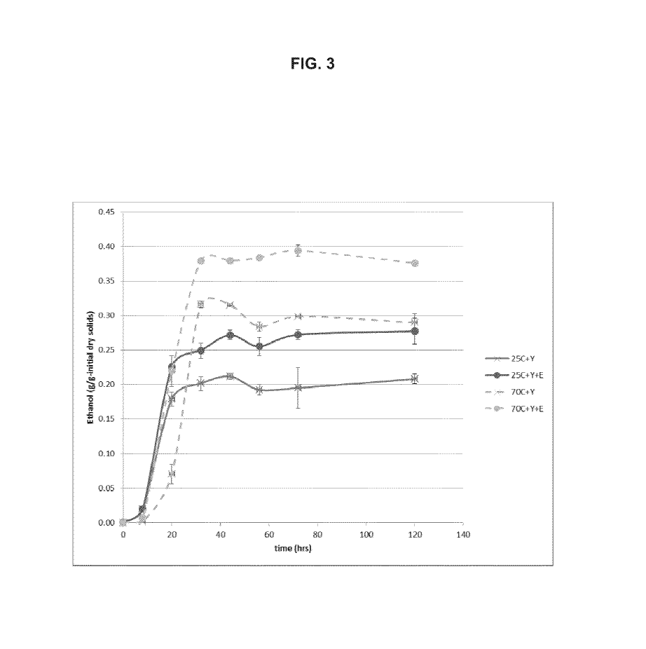

In FIG. 2 and FIG. 3, data labels preceded by a “25” or “70” refer to the preheating condition used. Samples with an “E” contain enzyme, “Y” contain yeast, and some may contain both or neither as indicated. A yeast and enzyme control is also included which has the same amount of yeast an enzyme used in the samples, but in 100 g YPD media in the absence of sugarbeet.

During fermentation, samples were collected at 0, 8, 20, 32, 44, 56, 72, and 120 hours by transferring 1-2 grams of sample into a new 15 mL centrifuge tube, weighing, and 10 mL of water was added. After mixing, samples were centrifuged at 8000 rpm for 10 min and the supernatants were stored at −20° C. for analysis. Sucrose and glucose concentrations were measured using a Y512700 Select Biochemistry analyzer and a Shimadzu HPLC with Biorad Aminex HPX-87H column operated at 60° C. with 0.05 mM H₂SO₄ mobile phase was used to measure ethanol, glucose, arabinose, glycerol, lactic acid, acetic acid, formic acid, and a combined xylose-galactose-mannose peak.

### Results and Discussion

### Effect of Initial Solids Content (10-20%) and Pretreatment Temperature

Actual solids contents were 9.5% and 19% solids in the raw beet, but descriptive labelling as 10% and 20% are maintained here for consistency. FIG. 2 and FIG. 3 shows the changes of ethanol contents over the course of the fermentation for various conditions at 10%, and 20% initial solids loading, respectively. Average values for duplicates are shown in all graphs, all duplicates showed relatively good agreement.

These results indicate that high theoretical SB conversion to ethanol in unsterile and non-supplemented conditions can be achieved within three days. Preheating to 70° C. for 2 hours reduces the lag time for ethanol production by approximately one day as compared to no-preheating (similar results are achieved in 2 days, rather than 3). As shown in FIG. 2 and FIG. 3, significantly more ethanol is produced in the time prior to the 32 hour time point in the sample derived from the heat sterilized/pasteurized sugar beets than in a sample identical except for the absence of the sterilization/pasteurization step.

Additionally, enzymatic hydrolysis improves liquefaction within 1 day and hydrolysis is evident with increased overall ethanol titer of about 5 g/L (˜30→35 g/L) for the 10% TS experiments. 0.3 g ethanol/g initial dry solids is produced for the yeast only samples, while 0.37 g/g initial dry solid (approximately 90% of theoretical) were produced with the SSF conditions tested.

pH dropped for all cases from 6-7 to about 4 within approximately 24 hours. Sucrose decreased to near zero within 48 hours for all conditions with either enzyme or yeast, indicating potential invertase activity in the enzyme preparation. For both SB controls which had no enzyme and yeast, sucrose decreased more gradually, with 25-40% remaining by day 5, with corresponding increases in glucose concentrations from hydrolytic cleavage of sucrose to glucose and fructose. The amount of glucose increase is only about half of that as would be expected from sucrose hydrolysis, indicating non-ethanologenic microbial activity is likely present. Maximum glucose concentrations were approximately 50% greater in enzymatically hydrolyzed samples.

As with the results for 9.5% TS sugarbeet, 19% TS sugar beet trials produce high levels of ethanol within 48 hours in a minimally controlled environment. Trials indicate that for pre-heated samples, ethanol production of approximately 0.3 g/g initial dry solid is achieved without the addition of enzymes, and approximately 0.4 g/g initial dry solid is achieved with enzyme addition. This upper value represents close to 100% of theoretical yield expected.

Multiple replicates of the ˜20% TS initial solids content achieved similar results for the SSF conditions (FIG. 3). A detailed analysis of component sugars and key fermentative products were evaluated. Notably, lactic acid is present at levels approaching 0.05 g/g, as well as acetic acid at ˜0.03 g/g, glycerol at ˜0.3 g/g and formic acid to a lesser extent. These non-ethanol fermentation byproducts are evidence of alternate pathways for utilization of carbon and largely indicative of microbial contamination primarily by lactobacillus species. Occurring prior to or during yeast fermentation. Also of note, residual glucose concentration in the 25C+Y+E condition is indicative of incomplete fermentation and arabinose (a 5-carbon sugar) is only remaining solubilized in the supernatant in the trials employing enzymes. It is not expected that the yeast can consume the arabinose, which is consistent with detection of the residual in the supernatant.

### Effect of Pretreatment Temperature and Microbial Contaminant Controls

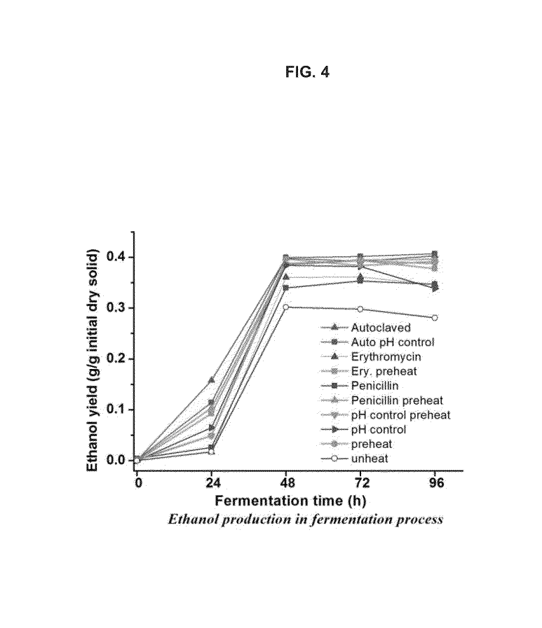

Ethanol yields for experiments testing various temperature, pH, and antibiotic treatments are shown in FIG. 4. It can be seen that pretreatment temperature affects the ethanol yield greatly. The two red lines indicate autoclaved SB which results in the highest ethanol yield, about 0.4 g/g of dry solid. Unheated SB had the lowest ethanol concentration, only 0.3 g/g of dry solid. The ethanol production of SB treated with erythromycin or penicillin, alone, was not as high as with the heated or pH reduced groups. Preheated and pH reduced groups did not show much difference in ethanol yield between 48-72 hours.

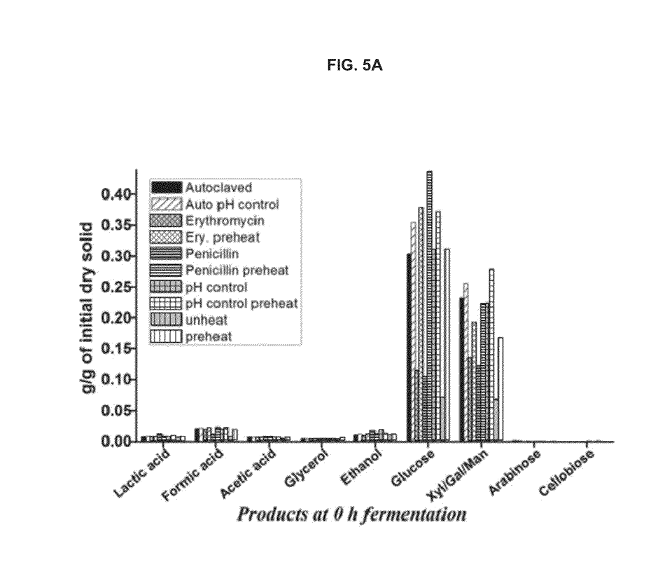

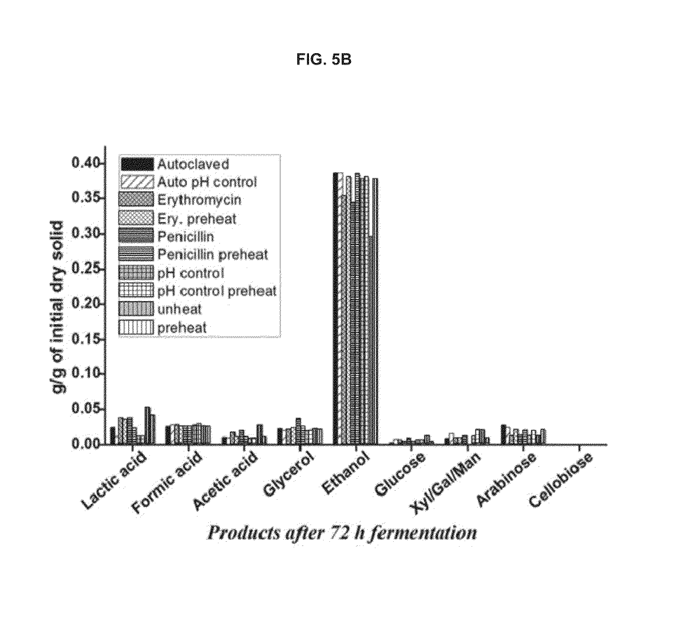

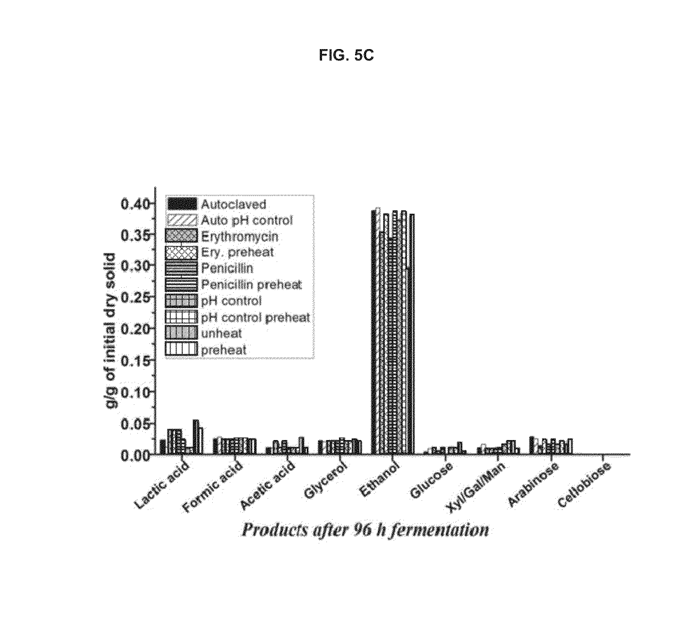

Byproducts and sugar concentrations in the fermentation are shown in FIG. 5. All pretreatment conditions (temperature, pH adjustment, antibiotic addition) reduced byproduct formation compared to control results. Initial pH adjustment to 3.5 most significantly reduced lactic acid production, and the combination of autoclaving with initial pH adjustment resulted in the highest ethanol yield of over 0.38 g/g initial dry solid at 96 hours. Higher temperature pretreatments tested here have higher final ethanol yields and lower lactic acid concentrations. Additionally, utilization of antibiotics was not superior to the pH or temperature pretreatment methods either alone or in combination with preheating at 70° C. for 2 hours. Also, a noticeable benefit in the rate of liquefaction was seen for the thermal pretreatment conditions

### Example 3

### Fermentation of Sugar Beet Roots Using a Continuously Stirred Reactor, Distillation, and Second-Stage Batch Bottle Fermentation

### Objective

Fermentation of ground sugar beet root for ethanol production at the 2 kg (fresh weight) scale, employing a 5 L stirred tank fermentation reactor, commercially available enzymes, and an industrial S. cerevisiae yeast (Bioferm XR), is described in this section. Removal of ethanol through vacuum distillation, and secondary fermentation using either S. cerevisiae or E. coli KO11 in shake-flask experiments are also described. The two stage fermentation experiments are aimed at a design of sequential enzymatic and biological conversion processes and optimization of operating conditions, thus resulting in an improvement of overall performance and economy of a system to produce ethanol. Here, enzyme and yeast loadings were the same as the previous batch shake-flask experiments. To evaluate the influences of different particle size and starting TS % of sugar beet on ethanol production at a larger scale, 4 trials of fermentation were carried out anaerobically at 37° C. with 50 rpm agitation after autoclaving.

### Methods

Methods for the bioreactor-scale experiments are similar to those used for the batch-bottle scale experiments reported in the previous section, with the following exceptions; Sugar beet root variety ECN115, rather than B4430R, was used and a 5-liter Bioflo 3000 fermenter (New Brunswick Scientific) used with 50 rpm agitation speed and anaerobic conditions. Enzyme loading is calculated according to the compositional contents of the sugar beet sample and was approximately equivalent to batch experiments (CTEC2, 67 FPU/g cellulose; HTEC2, 98 XU/g hemicellulose; NZ188, 34 CBU/g cellulose; pectinase, 65 PGU/g pectin). Also, due to foaming experienced in early tests, 1 mL of antifoam was added at the start of fermentation.

A laboratory scale distillation method was set up to remove and collect all the ethanol from the fermentation broth using a vacuum rotary evaporator (BUCHI Rotavapor). Approximately 500 mL of broth was filled into the evaporating flask and was distilled at 50° C. and 8-14 psia for 45 minutes. Most of the ethanol was removed from the stillage, along with significant water, and gathered in the collecting flask during this single stage distillation. Ethanol concentrations of the remaining stillage and condensed product were also measured by YSI analyzer.

In order to more fully use sugars left in the fermentation broth and maximize the ethanol production, a 2^{nd} stage of fermentations were performed using E. coli KO11 and S. cerevisiae (Bioferm XR), respectively, using the stillage obtained after distillation from trials A and B. The TS % and VS % of stillage were measured before use. Stillage was tested using the batch bottle fermentation methods described in the previous sections, with the exception of using E. coli KO11 as the biological catalyst following trial A. Enzyme loadings were also the same in the 1st stage fermentations as adjusted by initial solids content for the stillage. In order to focus on the effect of E. coli fermentation, two bottles were heated to 99° C. for 30 minutes to deactivate any yeast left in the stillage as a control. A fifth bottle was used as a stillage blank control with no treatment after distillation. For experiments using stillage from batch A, initial pHs were adjusted from 4.0 to 6.8-7.0 by adding 1.4 mL 10 M NaOH/bottle. Following batch B, S. cerevisiae was used to investigate the performance of additional yeast in a 2^{nd} stage fermentation and no pH adjustment was performed. Similar to the other bottle fermentation trials, 2^{nd} stage anaerobic fermentation was carried out at 37° C. with 150 rpm orbital shaking for 3 days. Duplicate samples were taken at 0, 24, 48, 72 hours and analyzed for ethanol concentration.

### Results and Discussion

### Simultaneous Saccharification and Fermentation (SSF) (1^{st} Stage Fermentation)

Details of the four fermentation trial conditions are shown in Table 6, which cover a range of experiments for both large and small particle sizes and initial solids content loadings approximately between 20% and 22% total solids.

TABLE 6
Conditions for 5 L Fermentation Trials

| | SB | TS | | |
|---|---|---|---|---|
| Trial | (g) | (%) | VS/TS | Description |
| A | 2100 | 19.8 | 0.969 | Fine grind |
| B | 1800 | 22.4 | 0.973 | Rough grind |
| C | 2050 | 21.9 | 0.973 | Fine grind |
| D | 2196 | 19.8 | 0.971 | Fine grind |

On average, about 2 kg of sugar beets were used in these fermentations. Autoclaved sugar beets in the fermenter visibly started to liquefy within 30 minutes in the mechanically mixed regions, with a majority of the beets in fermenter becoming liquefied within one day. Results for final (72-hour) ethanol production, TS %, and VS % are shown in Table 7.

Trials A and D, which had the same starting TS % of 19.8%, performed the best among these fermentations with ethanol yields of 0.44 and 0.43 g/g initial dry solids, respectively.

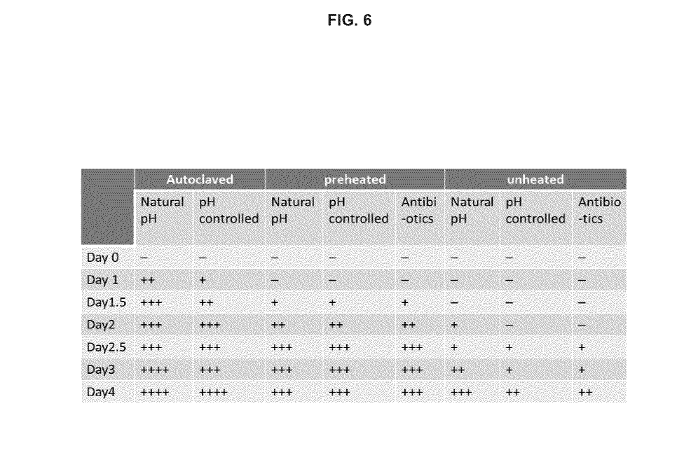

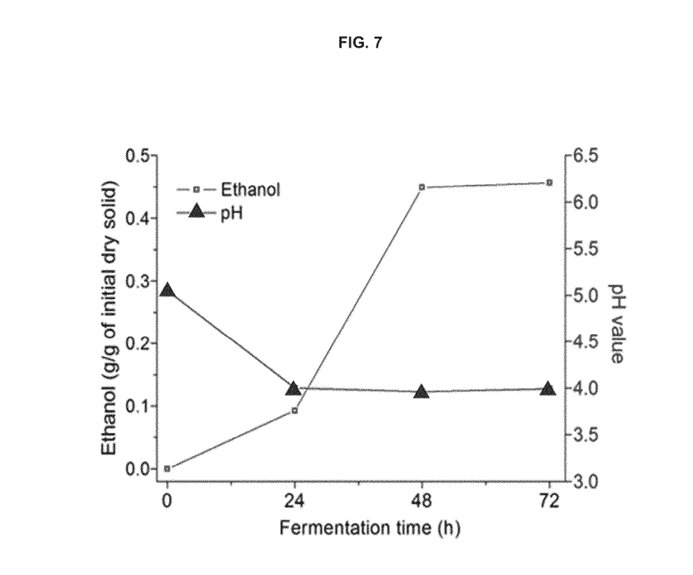

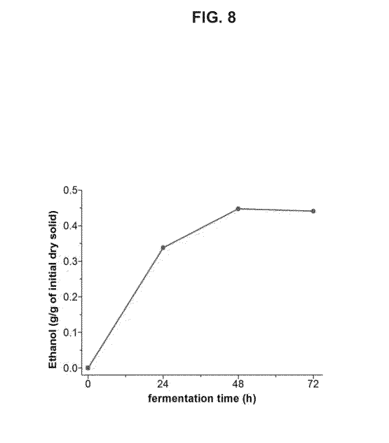

The total solids remaining (5.6%) and VS/TS ratio (0.88) after these two fermentations are very close. The ethanol productions of these two fermentations within 3 days are also plotted in FIG. 6. Ethanol production reached near maximum within 24-48 hours.

TABLE 7
Ethanol Production Results for Fermentation Trials

| | Sugar | | EtOH | EtOH | | |
|---|---|---|---|---|---|---|
| | Beet | TS | Yield | Conc. | Particle | 2-stage |
| Run | (g) | (%) | (g/g) | (g/L) | Size | Ferm. |
| A | 2100 | 19.8 | 0.44 | 87.4 | Fine | E. coli |
| | | | | | grind | |
| B | 1800 | 22.4 | 0.36 | 80.1 | Rough | Yeast |
| | | | | | grind | |
| C | 2050 | 21.9 | 0.37 | 81.8 | Fine | |
| | | | | | grind | |
| D | 2196 | 19.8 | 0.43 | 86.7 | Fine | |
| | | | | | grind | |

Trials A-D had varying initial solids contents from 19.8-22.4%. The ethanol yield of trial C was only 0.37 g/g and 85% of theoretical yield (Table 7). The solids remaining and VS/TS were higher than trials A and D. These results demonstrated that there were more fermentable components left in the broth, and the starting TS % may be very important to the ethanol fermentation. In trial B, a larger particle size was evaluated. Only 0.36 g/g of ethanol was obtained after 72 hours of fermentation, and the highest solid left (8.1%) among these trials. Moreover, yield was still increasing after 72 h fermentation and was higher at 96 hours. This indicates that beet with larger particle size may delay the fermentation rate and decrease the production efficiency likely due to mass transfer limitations.

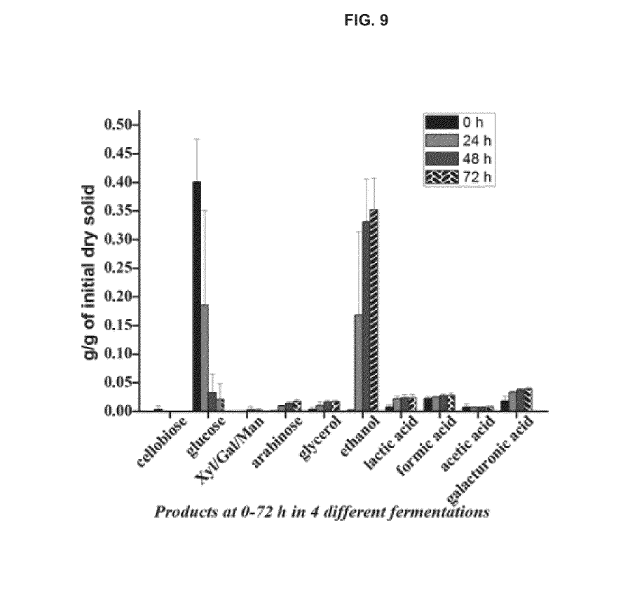

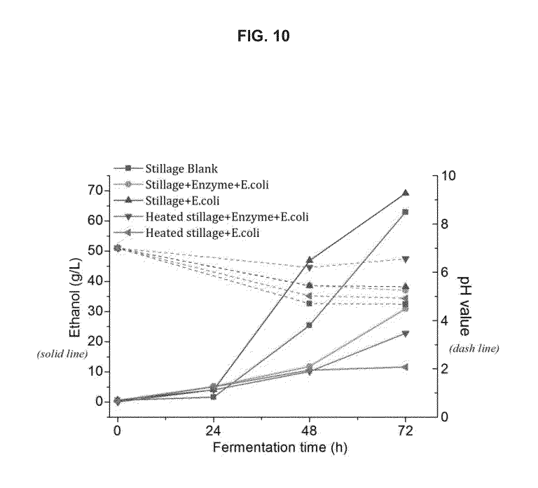

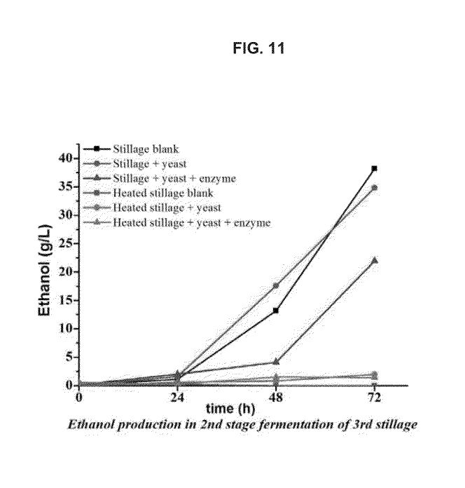

Results showed that components in these trials had similar trends within 3 days, as plotted in FIG. 9 for trials A-D. Notably, residual glucose concentration at 72 hours is indicative of incomplete fermentation and arabinose (a 5-carbon sugar) and galacturonic acid (a sugar-acid from pectin hydrolysis) were remaining solubilized in the broth. These components are not consumed by yeast, which is consistent with detection of the residual in the supernatant. Acetic acid was at level of 0.008 g/g and stable from 0-72 hours. Also lactic acid was present at levels gradually approaching 0.024 g/g, as well as formic acid at 0.028 g/g, and glycerol at 0.017 g/g.

### Example 4

It is known that sugar and other carbohydrates contained in biomass constitute a fundamental source for large scale food, fuel, and chemical production; however, material handling needs typically require that biomass be squeezed, extracted, or diluted to a practically manageable consistency. For example, sugar production from sugar beet and sugar cane is achieved primarily with a combination of hot water diffusion and mechanical pressing. The extracted sugar (sucrose) solution is further purified to table sugar or can be fermented to products such as alcohol. A residual pulp or bagasse is also created in the process. Similarly, industrial scale ethanol production from corn or other high starch content feedstocks generally begin with dilution of the feedstock to a desired concentration, followed by thermal and enzymatic liquefaction and hydrolysis steps prior to fermentation. Sugar beet roots, while perhaps only containing 20-25% solids (75-80% moisture), are largely hygroscopic due to their high sugar and pectin contents (Table 8), and if ground, will require significant water addition in order to effect a reduction in viscosity.

TABLE 8
Example Sugar Beet Root Composition (UCD Varieties, 2012)

| | WET BASIS | (std. dev.) | DRY BASIS | (std. dev.) |
|---|---|---|---|---|
| Moisture | 79.4 | 1.5 | | |
| Ash | 0.6 | 0.1 | 3.0 | 0.3 |
| Soluble Sugars | | | | |
| Sucrose | 14.7 | 1.7 | 71.1 | 3.3 |
| Glucose | 0.1 | 0.0 | 0.3 | 0.1 |
| Fructose | 0.0 | 0.0 | <0.2 | 0.1 |
| Cellulose | 1.1 | 0.2 | 5.5 | 0.7 |
| Hemicellulose | 1.0 | 0.1 | 4.9 | 0.6 |
| Lignin | 0.2 | 0.0 | <1 | 0.0 |
| Pectin | | | | |
| GalA | 2.4 | 0.3 | 11.8 | 1.0 |
| Protein | 0.8 | 0.1 | 3.8 | 0.6 |
| TOTAL | 100.4 | | 101.6 | |

Described herein is a novel method and system comprised of processes for efficient conversion of lignocellulosic biomass that have high contents of sugar and pectin, such as sugar beets, without the need for additional water and significant preprocessing. This method might also be used for feedstocks such as casava, potato and sweet potato, or other fruits and vegetables and/or wastes generated during collection or processing of these agricultural products. This method describes operation in either a Separate Hydrolysis and Fermentation (SHF) configuration, or Simultaneous Saccharification and Fermentation (SSF) configuration. After biomass pretreatment involving, washing, size reduction, and thermal and/or chemical pretreatment steps, enzymatic liquefaction and hydrolysis is either achieved in a first stage vessel with high solids mixing design, or in the fermentation vessel. For the SHF design, when viscosity is reduced below a determined level in the liquefaction vessel, the contents are transferred to the first stage fermentation reactor optimized for liquefied substrates and allowing for additional enzyme, microorganism and chemical addition as required. After or during fermentation, residual solids can be removed if desired and fermentation products separated from the fermentation broth using traditional distallation or separation processes for downstream recovery. A second stage fermentation can be emoployed with conditions optimized for conversion of residuals from first stage fermentation alone, or in combination with additional pre-treated beet leaves.

Table 9, as well as FIG. 12-FIG. 17, describe the operational configurations of an exemplary system and functional aspects of the various unit operations.

TABLE 9
Description of Operational Configurations for Exemplary System

| Operational | | Liquefaction | Fermentation 1 | Fermentation 2 |
|---|---|---|---|---|
| Configurations | Pretreatment | and Hydrolysis | and Distillation | and Distillation |
| Functional | Wash | Mix | Mix | Mix |
| Descriptions: | Grind | Maintain | Maintain | Maintain |
| | Preheat (25-120° C.) & | Temp (25-70° C.) | Temp (25-50° C.) | Temp (25-50° C.) |
| | Cool | Enzyme | Fermentation | Fermentation |
| | [Optional] | Addition | Organism | Organism |
| | Chemical | Microbial | Addition | Addition |
| | Addition (for | addition | Enzyme | Additional |
| | pH or | [Optional] | Addition | Feedstock |
| | Microbial | | [Optional] | Addition |
| | Control) | | Solids | [Optional] |
| | [Optional] | | Separation | Enzyme |
| | | | [Optional] | Addition |
| | | | Distillation or | [Optional] |
| | | | Product | Solids |
| | | | Removal | Separation |
| | | | | [Optional] |
| | | | | Distillation or |
| | | | | Product |
| | | | | Removal |
| 1—(SSF 1) | Roots and/or | | Pretreated | |
| | Leaves | | Feedstock | |
| 2—(SSF 2) | Roots and/or | | Pretreated | Fermentation 1 |
| | Leaves | | Feedstock | Stillage |
| 3—(SHF 1) | Roots and/or | Pretreated | Liquefied | |
| | Leaves | Feedstock | Feedstock | |
| 4—(SHF 2) | Roots and/or | Pretreated | Liquefied | Fermentation 1 |
| | Leaves | Feedstock | Feedstock | Stillage |
| 5—(SSF 1 + 2) | Roots and | | Pretreated Roots | Pretreated leaves |
| | Leaves, | | | and Fermentation |
| | separately | | | 1 Stillage |
| 6—(SHF 1 + 2) | Roots and | Pretreated Roots | Liquefied Roots | Pretreated Leaves |
| | Leaves, | | | and Fermentation |
| | separately | | | 1 Stillage |

Beet grind size has been determined to be an important parameter in overall system performance. A laboratory food processor is used to reduce particle size to the range of 1-10 mm diameter, although a grinder or macerator could be used at industrial scale.

Operation of the hydrolysis and fermentation reactors can be batch, fed-batch, or continuous, however, the size of the hydrolysis reactor will be smaller than the fermentation reactors as residence time in the first stage reactor are only sized for sufficient liquefaction. Reaction conditions in the hydrolysis reactor will be from 25° C.-120° C. with the ability to add liquid preparations of chemicals and enzymes to the reactor upon loading the reactor with feedstock or during mixing operation. Cooling, in the form of an external heat exchange jacket is envisioned in practice. Yeast and enzymes will not be added until the temperature of the reactor and reaction mixture is cooled to suitable temperature.

The enzymes used to liquefy the substrate must be appropriate for the substrate. Here, commercial preparations of cellulases (C), hemicellulases (H), β-glucosidases (B), and pectinases (P) are used (Novozymes CTEC2, HTEC2, Novo188, and Pectinex Ultra SP-L). Pectinases alone may not be sufficient to effect liquefaction and a combination of cellulases with pectinases and/or hemicellulases and yeast achieve the most rapid and complete liquefaction conditions. Further optimizations of enzyme loading conditions are being investigated.

### Example 5

### Enzymatic Hydrolysis and Viscosity Reduction of Ground Sugar Beet Roots

### Objective

Reduction in apparent viscosity (liquefaction) can occur during processing of beets via a combination of thermal and biochemical mechanisms. In order to illustrate the effect of preheating and enzyme selection and loading on beet liquefaction during hydrolysis, a rotational rheometer setup with stirrer designed to accommodate solid particles can be used to track changes in apparent viscosity.

### Methods

Similar to as described in the previous example, fresh washed beets were ground using a food processer to achieve a maximum particle size of around 5-10 mm in diameter. Ground beets were then passed through a manual food mill with 5 mm diameter screen opening to ensure a maximum particle size cutoff. Beets were either autoclaved (121 C, 20 minutes) or not, depending on the sample conditions selected. Initial total solids contents were measured as described previously, and distilled water added to bring the initial solids contents down to approximately 20%. For all samples tested, this ranged from 40-50 mL of water required to be added to 200 g of fresh or autoclaved beets.

An Anton-Parr RheolabQC rotational rheometer (torque range 0.075-50 Nm), equipped with an open paddle stirrer (Model ST59) designed for testing of building materials with solids up to 5 mm in size was operated at a continuous fixed rotation speed of 50 revolution/second (approximate shear rate 50 s⁻¹). Beets were tested in wide-mouth 500 mL glass jars (Ball Corporation) and a special form made to ensure identical placement of the stirrer in the jars between samples. An electric, thermostatically controlled heating jacket was used to maintain sample temperature at 50 C during hydrolysis for 24 hours or until the minimum torque was reached.

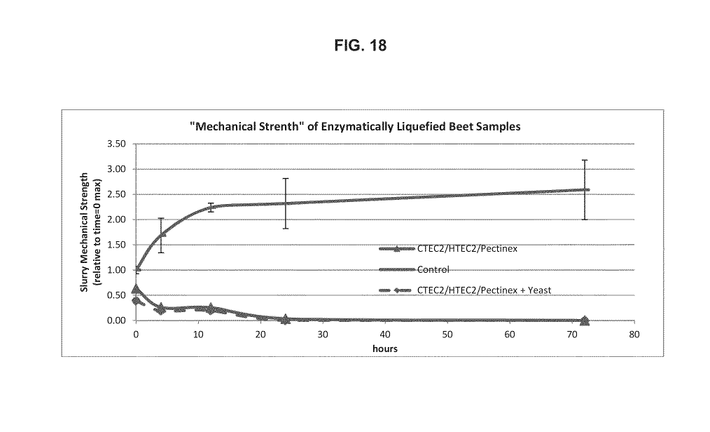

Novozymes products, CTEC2 (Cellulase, 120 FPU/mL), HTEC2 (Hemicellulase, 131 XU/mL), and NS22119 (Pectinase, 10007 PGU/mL) were added at rates of 0.7, 0.07, and 0.05 mL per 100 gram of beet substrate, respectively. A reduced condition with 0.07 mL CTEC2, and the same amount of HTEC2 and NS22119 is also shown, and referred to as 0.1×CTEC2×HTEC2×Pectinase in the data label for FIG. 18. Additionally samples with 0.05 mL pectinase per 100 gram of beet substrate were also tested.

### Results and Discussion

As tested with this apparatus, a readily pourable liquid with low yield stress might have a viscosity around 1000 cP, while water would register <100 cP. As shown in FIG. 18, ground non-autoclaved beets have a high initial viscosity which does not drop substantially over 24-hours. Autoclaving alone reduces the initial apparent viscosity by approximately half. For the non-autoclaved beets, pectinase addition effected a significant reduction in viscosity over 24-hours, although still maintained some semi-solid character and obvious yield stress. Addition of the multi-enzyme cocktail resulted in liquefaction to below 1000 cP in approximately 10 hours. Autoclaved beets with a similar amount of enzyme addition achieved the same viscosity in approximately 1 hour, or even 2 hours with the ten-fold reduction in cellulase usage.

### Example 6

### Pilot Scale Tests (Task 4.2)

### Introduction

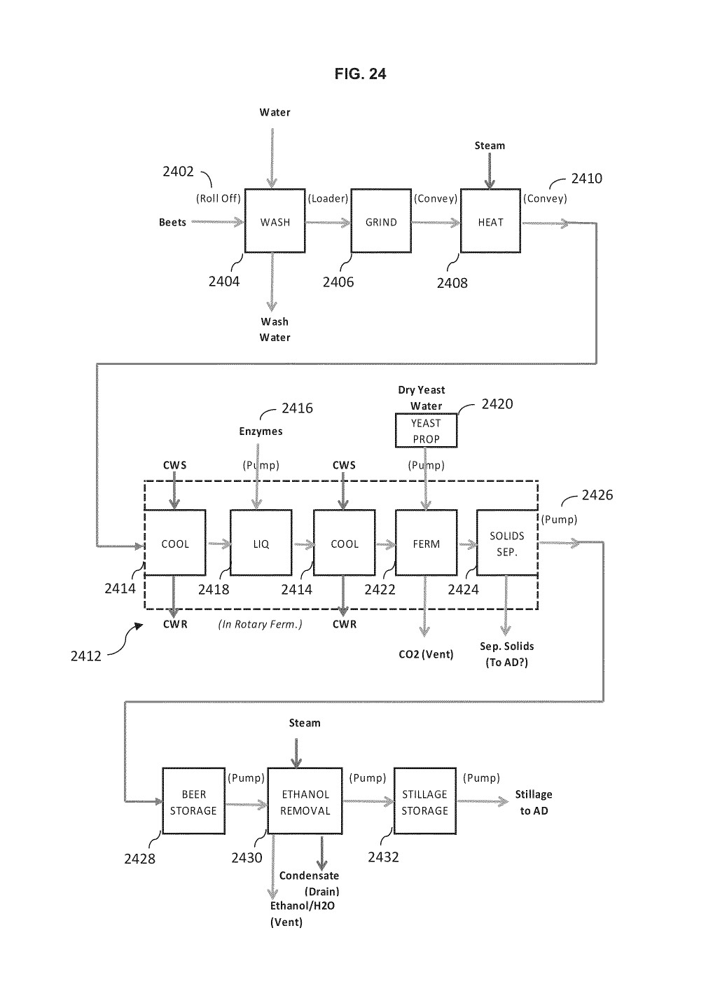

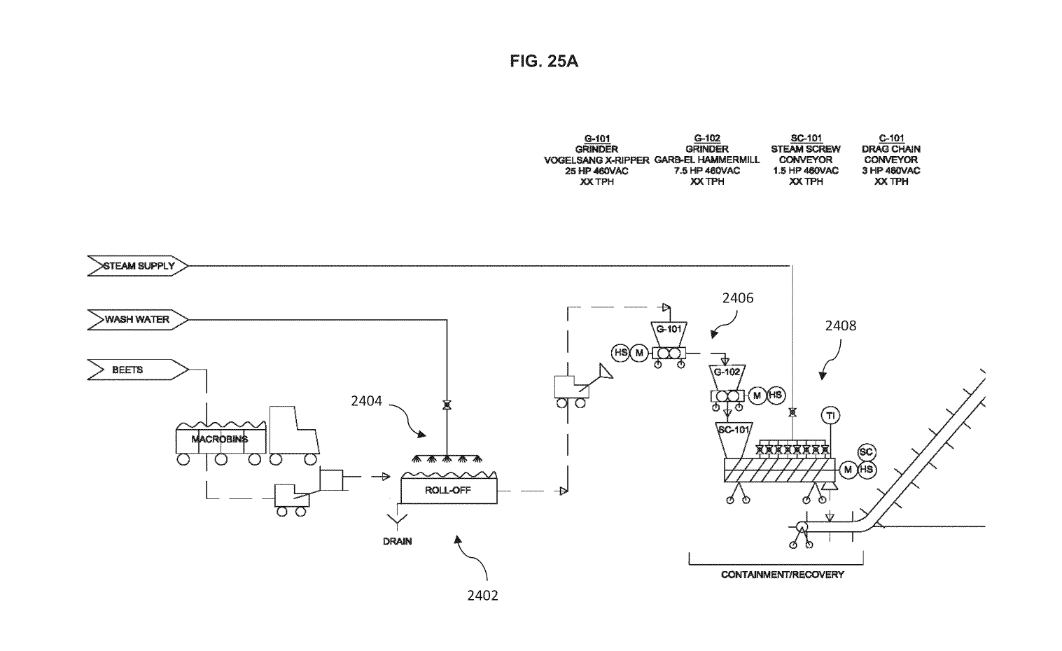

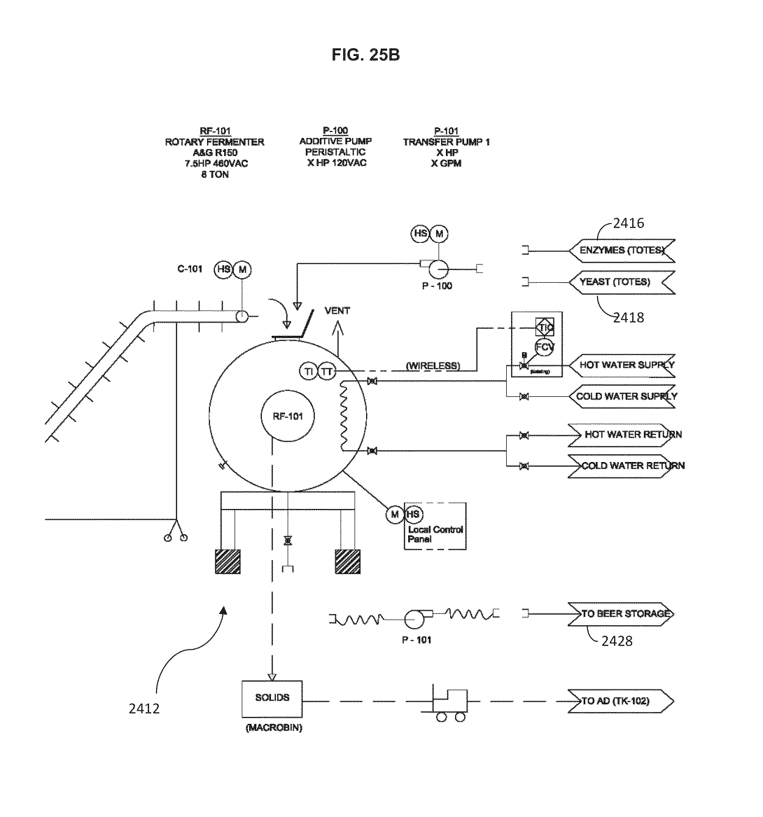

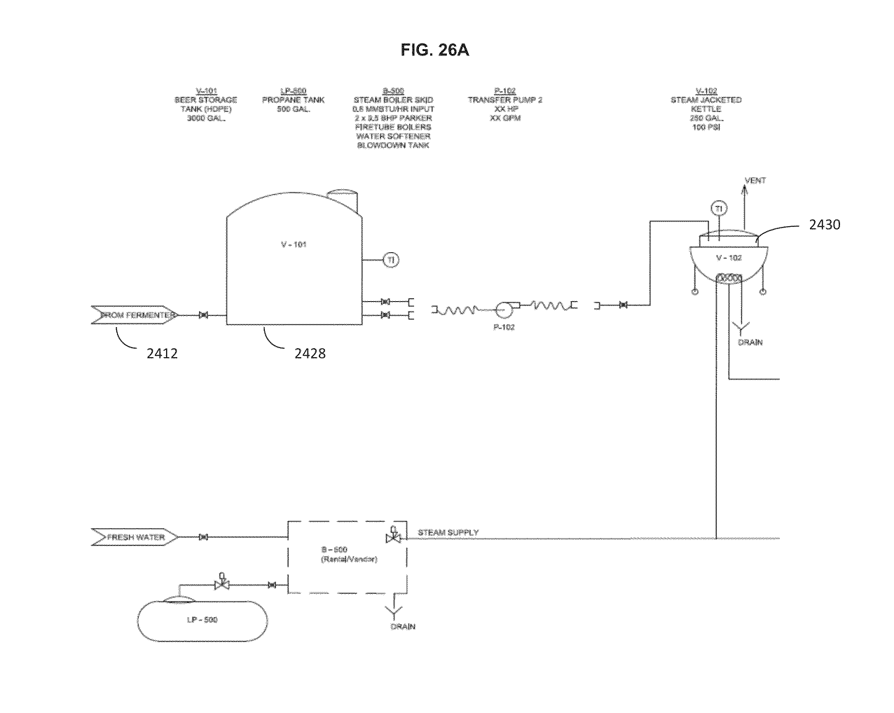

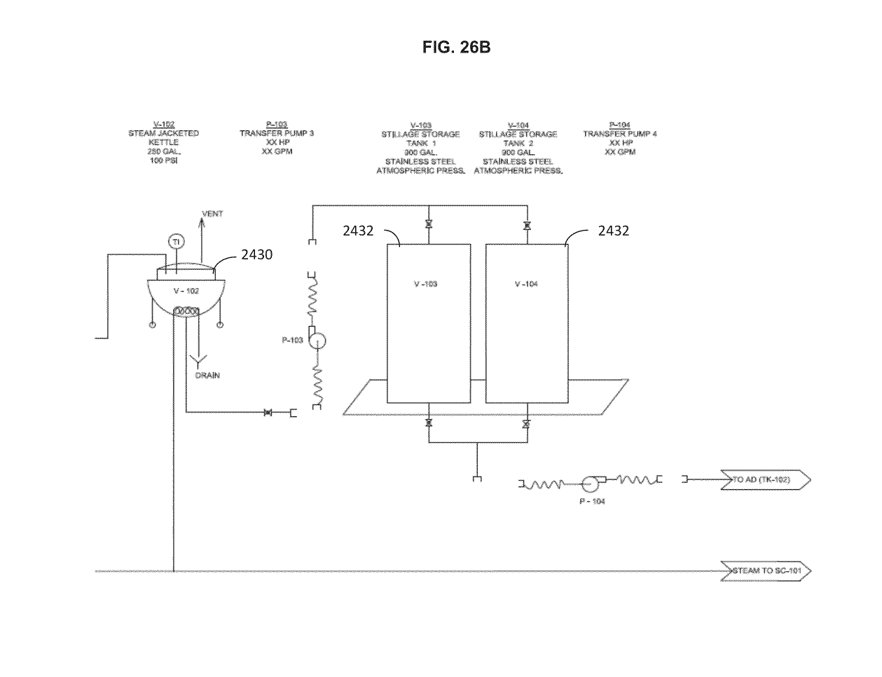

A pilot-scale demonstration of the novel sugar beet bioethanol process developed in the laboratory and described previously was conducted at the UC Davis Biogas Energy Project facility using beets grown and harvested on campus. Harvest and storage, processing, fermentation, ethanol removal and anaerobic digestion operations were performed between October, 2012 and January, 2013 as described in more detail to follow. Approximately 40 tons of beets were processed in total and results from triplicate 5-ton batches are reported here to characterize performance metrics for ethanol and biogas production for the pilot process. Average results of 0.36 gram-ethanol per gram-initial total solids (range, 0.33-0.39) and 23 gallons-ethanol per wet-ton of beets (range, 20-26) were achieved. Block-flow and process-flow diagrams describing the process implemented are shown in the subsequent section 3.6 with initial and actual mass balance metrics observed, see, for example, FIGS. 24-26.

Sugar beet cultivars EGC184 and ECN115 provided by KWS Betaseed were grown on a 2-acre plot at UC Davis by the Plant Science Department Field Operations Division under the supervision of Dr. Steve Kaffka and Mr. Jim Jackson and in coordination with Mendota Bioenergy, LLC. Planting was performed from seed in June, 2012 and fertilized at the rate of 120 lb-N/acre. Harvesting was accomplished first by defoliation of the beets using a mechanized rotating rubber-flail beater pulled by a tractor with beet leaves left in the field. Defoliated beet roots were removed manually using beet-knives and further removal of any leaf material using the knives was performed prior to placement of the beets into macro-bins for transportation either directly to the processing site or cold-storage as required. Cold-storage rooms (34° F.) capable of storing approximately 10-tons of beets located at the UC Davis Robert Mondavi Institute (RMI) Food Science Pilot Plant were utilized. For all processing trials, equal amounts of each beet cultivar were used as a composite mixture.

For each process batch, beets transported to the site in macro-bins were unloaded individually into a 20-yd³ roll-off trailer modified with a false bottom containing sections of chain-link fencing (2402). Beets were spread over the screens and washed (2404) manually.

Cleaned beets were then piled into a second roll-off trailer, which for trials 4-7 was outfitted with a 4″ steam hose with perforations running along the bottom of the trailer. For these trials, once the roll-off was filled to the desired level, a heavy duty tarp was pulled over the top of the trailer and 40-psi saturated steam was delivered through the hose to the bed of beets for 2-hours. This process, termed “pre-steaming”, was implemented to achieve two purposes; firstly to reduce inherent microbial populations on the surface of the beets prior to grinding, and secondly, to raise the initial temperature of the beets entering the steam-injection process in order to achieve a higher final exit temperature. Pre-steaming of beets was followed by loading into a ⅓-yd³ bucket loader for delivery to the grinder for subsequent processing.

The overall processing scheme employed consisted of two grinding steps (2406), direct injection of steam for heating of the feedstock (2408), and transportation of the heated ground beets (2410) using a drag-chain conveyor to approximately 14′ elevation for loading into the subsequent fermentation vessel.

A combination of two grinders were employed to achieve the desired maximum particle size of around <¼″ as determined necessary from previous experiments. Two grinders were used here due to equipment availability and throughput requirements; however a single piece of equipment could likely be used for other similar applications. The first grinder consisted of a twin-shaft macerator (Vogelsang X-ripper) with hardened stainless steel teeth driven by a 25 HP motor and nameplate capacity of 25-tons per hour. This grinder was elevated approximately 5′ above the ground and fitted with an inlet receiving hopper capable of holding approximately ⅓-yd³. Beets were metered into the hopper from an elevated loader bucket and reduced in size to approximately 1″ pieces. The second grinder consisted of a hammer mill (Garb-el) with ¾″ screen openings that was situated directly under the first grinder to allow gravity feed from the first unit to the second. A 1.5 HP fixed-speed feeder screw delivered beets to the 7.5 HP hammer mill drive motor at approximately 2-tons per hour to result in approximately <¼″ particles.

Once milled, ground beets fell into the inlet of a flighted screw conveyor, custom modified to allow direct injection of steam into the ground beets, as well as elevate beets enough to allow gravity delivery onto the next conveyor. As such, the conveyor shaft operated at approximately 15° from horizontal. The stainless steel conveyor overall length was 8′ with solid helical flights having an 8″ pitch and removable top cover. The distance from center of the inlet receiving area to the center of the outlet delivery area was approximately 6′. A 1.5 HP variable speed drive was used to accommodate a federate range from approximately 1-6 tons/hour during operation. A central 2″ carbon-steel steam manifold was reduced to a header containing eight 1″ EPDM low pressure steam-hoses capable of delivering steam directly to 1″ full-port ball valves located in four opposite equally spaced locations on each side of the conveyor. Steam pressure and flow could be modulated manually through throttling of the injection valves as needed. The outlet temperature of the steamed beets were monitored with an RTD insertion probe and recorded manually. Once steamed, beets were delivered to a 30′ section of a retrofitted 3 HP drag-chain conveyor (SMC), which operated a fixed speed and elevated the beets to approximately 14′ for delivery to the fermenter.

To provide steam for sanitation and process heating requirements, dual 9.5 bHP low-pressure steam boilers (Parker: 400,000 btu consumption/hr each) preassembled on a skid with water softener, treatment chemical addition tank and pump, make-up and blow-down connections, and all required accessories was rented from San Jose Boiler Works, San Jose, Calif. for the duration of the trial. Maximum boiler steam pressure rating was limited to 80 psig, but operated between 20-40 psig. A 2″ 50′ section of EPDM low pressure steam-hose was used to deliver steam from the boiler to a single point-of-use and outfitted with a fiberglass-insulated, silicone impregnated, safety sleeve for personnel protection. Boilers use was dedicated to one task at a time (i.e. beet heating, fermenter sanitation, beer heating, etc.), and equipment usage scheduled accordingly.

A horizontal rotary fermenter (A&G Engineering) was selected as the primary reactor to be used for additional heating, cooling, and fermentation (2412) of the processed beet feedstock. The 8-ton capacity unit was purchased used from a winery in Napa Valley, Calif. complete with 7.5 HP motor and Programmable Logic Control (PLC) system (Allen-Bradley) for rotation and position control via proximity switches. The fermenter was mounted on a flat-bed trailer to allow mobility during processing, loading, and movement into place for fermentation near appropriate utility hookups. The dimensions of the fermenter are approximately 7.2° internal diameter with an 11.8° barrel length and 15.6° overall length including the conical end sections. The unit is equipped with a spring-loaded vent that opens when the vent location is rotated to the top. Rotation can be performed in either clockwise or counter-clockwise directions and operates at a fixed speed of approximately 1.3 rpm. Modification to the PLC program was performed to allow continuous mixing and automatic reversal of mixing direction. Mixing is achieved internally via movement of materials by a helical flight of approximately 1′ width welded to the inside of the fermenter wall. Rotation of the fermenter in one direction (“mixing”) moves materials towards the back wall, while rotation in the other direction (“unloading”) moves materials towards the front-conical section, which, if the end cap is removed, allows emptying of contents into a bin. During trials 4-7, continuous rotation was performed in the “mixing” direction for cooling and fermentation phases.

A wireless RTD temperature transmitter (Omega) was installed in a 6″ internal-projection thermo-well and receiver with 4-20ma analog output employed to allow data collection via the existing Biogas Energy Project facility PLC data-acquisition system. Heating and cooling of the fermenter contents were achieved via the dimpled external heating jacket covering approximately ⅓ of the fermenter barrel. Set-point temperature for fermentation was 37° C. Heating water at 70° C. was used in the jacket, supplied from the digester heating loop at the Biogas facility, and returned to the boiler in through the closed loop system. To achieve cooling (2414), 25° C. No. 3 process water from wastewater facility was used in a once-through configuration with disposal to the facility collection drain.

A 24″×20″ re-sealable man-way opening on top of the fermenter was used for loading, addition of enzymes (2416) and yeast (2420), and periodic sampling using a 1-liter sample container with an 8′ handle. Full length screens with approximately ⅛″ opening widths, covered the bottom of the fermenter and prevented large solids from leaving the vessel upon draining Access to the top of the fermenter was provided by a ladder and platform with railings.

During loading of the beets, hydrogen peroxide (3% H₂O₂ solution) was added at a rate of 5 ppm using a peristaltic pump dosed onto the beets leaving the conveyor and entering the fermenter. The speed of the pump was modulated to achieve a dosage rate of approximately 1 L peroxide per wet ton of beets and the purpose of this addition was to temporarily suppress unwanted microbial activity during the loading process.

Once beets were loaded into the fermenter, since the target final heating temperature (100° C.) had not been reached as was the case for trials 4-7, additional steaming of the beets was achieved by connecting the steam hose directly to the bottom drain of the fermenter and steaming the static-bed of beets until the target temperature was reached. Cooling of the beets (2414) was then performed quickly by disconnecting the steam line and connecting the cooling lines to the fermenter jacket and applying cooling.

In preparation for yeast addition (2420), yeast cells were hydrated by preparing 1 kg of dry yeast (Bioferm-XR™ by NABC) per ton of wet beets. Hydration was performed in clean 5-gallon plastic pails using distilled water pre-heated to 37 C. Yeast was added to the water and mixed thoroughly and allowed to incubate for between 30-60 minutes prior to addition to the fermenter (2422). Enzymes provided by Novozymes were utilized for hydrolysis (2418) and included a cellulase-rich product (Cellic CTEC2) a hemicellulase-rich product (Cellic HTEC2) and a pectinase-rich product (N522119). Standard activities for each enzyme are shown below in Table 10. Cellulase activity is reported on the basis of Filter-paper-units (FPU) as measured by the method reported by (Ghose 1987). Hemicellulase activity is reported on the basis of Xylanase-Units (XU) as measured by the method reported by (Ghose and Bisaria 1987). Pectinase activity is reported on the basis of Polygalacturonase units (PGU) as measured by a modified method as that reported by (Fernandez-Gonzalez, Ubeda et al.).

TABLE 10
Enzyme activities and target loadings

| Enzyme Stock | | | | Target Loading |
|---|---|---|---|---|
| Activities | FPU/ml | XU/ml | PGU/ml | (volume/mass) |
| Cellulase - | 125 | 5286 | n/a | 7 liters/ton initial wet beet |
| CTEC2 | | | | |
| Hemicellulase - | 74 | 9685 | n/a | 0.7 liters/ton initial wet beet |
| HTEC2 | | | | |
| Pectinase - | n/a | n/a | 10007 | 0.5 liters/ton initial wet beet |
| NS22119 | | | | |

Addition of yeast (2420) and enzyme (2416) to the fermenter was performed by direct addition through the top man-way while rotation of the vessel was stopped. Sampling, which occurred at 24, 48, 72, and 120 hours post enzyme and yeast addition, was also performed through the top man-way while rotation was stopped.

Upon completion of batch fermentation (2412) at 120 hours post enzyme and yeast addition, contents of the fermenter were pumped out (2426) of the fermenter to a 3000 gallon white HDPE holding tank and the volume of the “beer” recorded by direct measurement of the height in the cylindrical tank (2428). Solids that were retained by the screen were then emptied from the tank into a macro-bin by rotation of the fermenter in the “unloading” direction with the end-cap removed (2424). These solids could then be weighed and a sample saved for analysis.

Since a full scale distillation and ethanol recovery system could not be implemented for this project, an ethanol vaporization and removal system employing a 250-gallon steam-jacketed kettle (100 psig max. steam rating) was used (2430). An additional copper coil consisting of 50′ of ⅝″ tubing wound in multiple 3′ diameter passes was installed internal to the kettle volume to increase heat-transfer surface area for later trials. Heating of beer was performed in 200-gallon batches or less and heated until the temperature reached 100° C. as measured by an RTD probe with local digital temperature display. Once the target temperature was reached, cooling was achieved by passing facility cooling water through the jacket and coil until the temperature was below 75° C. The stillage was then transferred via a 2″ air-operated diaphragm pump (Warren-Rupp) to one of two parallel 900-gallon stainless steel storage tanks (1800-gallons total storage) for further cooling and storage until needed for feed to the anaerobic digestion system (2432).

Only trials 4, 5, and 7, were processed to remove ethanol and create a stillage product. Due to the several smaller volume batches required to remove ethanol, the stillage collected in the storage tanks were treated as a composite sample after completing all attempts at ethanol removal in the kettle.

Prior to all processing and sampling, attention was paid to sanitation of necessary equipment as follows. All previously used equipment was thoroughly washed with No. 3 process water and scrubbed with brushes as best possible to remove obvious debris. A high foaming, acid anionic, non-rinse sanitizer (Star-San™, Five-Star chemicals) was then prepared using 30 ml/gallon preparation dilution and used to completely rinse the process equipment including the grinder, hammer-mill, screw-conveyor, chain-conveyor, fermenter, beer storage tank, kettle, stillage storage tank, and all pumps and sampling equipment. Additionally, for the fermenter, additional steam sterilization was attempted by directing live-steam into the vented fermenter for a period of 30-minutes prior to use but after sanitation.

For collected samples, total solids determinations were made by drying in a 105° C. oven overnight, and volatile solids determine by further drying at 550° C. for 3-hours. All samples were prepared for soluble carbohydrate and ethanol analyses by dilution of ˜1-gram of slurry sample (weights measured accurately to determine proper dilutions) in 10-grams of distilled water into a 15-ml Falcon-tube, mixing (vortex) and allowing equilibrating for 10-minutes, followed by centrifugation at 8000×g for 10-minutes. The supernatant is then removed and filtered through a 0.22 μm filter into a 1-ml borosilicate glass HPLC vial. 1M H₂SO₄ is added to a final concentration of 0.05M and samples are frozen at −20° C. until ready for analysis. High performance liquid chromatography (HPLC) was carried out to test the content of ethanol, glucose, cellobiose, arabinose, glycerol, formic acid, acetic acid, lactic acid, galacturonic acid, and xylose/galactose/mannose/fructose as a cumulative peak. The Shimadzu HPLC-10ATVP HPLC and Aminex HP-87H column with RID and PDA detectors were operated with continuous sulfuric acid mobile phase (5 mM; flow rate, 0.6 mL/min) and oven temperature at 60° C. Amounts were quantitated by applying four-point external standard calibration curves. Sucrose concentrations were measured using a YSI 2700 Biochemistry analyzer equipped for dual sucrose and glucose determination.

### Results and Discussion

Several large scale trials were carried out during the pilot testing period with beets harvested 4 times during the pilot testing period as shown in Table 11. Approximately 25 of the 40 tons of beets harvested were used for process testing, troubleshooting, and optimization or were used in trials where process failures resulted in poor batch results. Initial trials attempted a Separate Hydrolysis and Fermentation (SHF) process configuration whereby hydrolysis and liquefaction were performed at 50° C. prior to cooling to 37° C. for addition of yeast. However, the extended time prior to yeast inoculation resulted in high microbial contamination rates and lactic and acetic acid levels greater than the final ethanol levels (data not shown). In an industrial non-sterile environment, time-to-yeast addition is an obvious critical variable as was also illustrated with trial 5, where even though a Simultaneous Saccharification and Fermentation (SSF) process configuration was employed, mechanical problems with the fermenter motor prevented proper temperature stabilization and delayed yeast inoculation, resulting in low final ethanol concentrations as well.

Three replicate trials of 5 tons each (Trials #4, 5, and 7), were considered successful demonstrations at the pilot scale and detailed operational and chemical analyses of these runs are presented further below. However, operational experiences and problems encountered during all trials are described so as to illustrate what difficulties arose and informed the overall decision making process.

TABLE 11
Sugar Beet Bioethanol Trial Summary Descriptions

| | | | | | | Time | | | |
|---|---|---|---|---|---|---|---|---|---|
| | | Initial | | | Yeast | to | Solids - | Solids - | Ethanol |
| | | Mass | Harvest | Configuration | Loading | Yeast | Ground | Steamed* | Max |
| Trial # | Trial Date | (tons) | Date | (SHF/SSF) | (mg/gTS) | (hrs) | (% wb) | (% wb) | (g/L) |
| 1 | Oct. 15, 2012 | 1.5 | Sept 17 | SHF | 0.5 | 48 | 19.8 | 19.1 | n/a |
| | | | (CS) | | | | | | |
| 2 | Oct. 22, 2012 | 7.7 | Oct 5 (CS) | SHF | 0.5 | 48 | 21.0 | 19.6 | 0 |
| 3 | Oct. 29, 2012 | 10 | 29-Oct | SHF | 0.5 | 48 | 20.6 | 19.6 | 23 |
| 4 | Nov. 13, 2012 | 5.4 | 13-Nov | SSF | 5.0 | 6 | 20.3 | 20.8 | 73 |
| 5 | Nov. 19, 2012 | 5.4 | 13-Nov | SSF | 5.0 | 6 | 20.7 | 19.6 | 77 |
| 6 | Nov. 26, 2012 | 4.9 | 13-Nov | SSF | 5.0 | 24 | 22.2 | 20.5 | 22 |
| | | | (CS) | | | | | | |
| 7 | Dec. 03, 2012 | 5.1 | 13-Nov | SSF | 5.0 | 6 | 21.6 | 20.0 | 87 |
| | | | (CS) | | | | | | |

CS = Cold Storage,
SHF = Separate Hydrolysis and Fermentation,
SSF = Simultaneous Saccharification and Fermentation

### Harvest, Storage, Washing, and Pre-Steaming

The process of harvesting and washing beets was extremely labor intensive. Harvesting required approximately 4-6 laborers to harvest around 2-tons/hour and approximately 2-3 laborers were needed to wash beets at a rate of 2-tons/hour. Both of these processes can be greatly automated to reduce labor input at larger scales. Trials 4-7 were all performed with beets harvested on the same date. Beets for trial 4 were used immediately, while beets for trial 5 were stored outside during the cooler fall weather. Beets for trials 6 and 7 were stored in the RMI cooler until ready for use. Growth of some grey-mold was evident on wounded areas of beets placed in cold storage for over 1 week.

Water use for washing was not measured but is estimated to have been approximately 100 gallons/ton. Washing by hand removed approximately 3% of the mass received at the plant in the macro bins through loss of soil and debris as measured for Trial #1. Other than removal of debris and mold, no obvious damage to the beet tissue was observed during handling and washing.

The pre-steaming process resulted in some beets that were closest to steam source discoloring to a dark grey or black color on the outside of the beet penetrating 1-2 inches into the tissue. Beets more than 2 feet from the steam source did not discolor. Overall, pre-steaming for 2-hours achieved an increase in bulk beet temperature from 20-25° C. to approximately 35° C. A small amount (˜1 L/ton) of condensate was produced during preheating which was dark brown/black in color and was not recovered.

### Grinding and Milling

No mechanical problems were experienced with the first grinder, however during Trial #2, the hammer mill experienced mechanical overload due to blinding of the screen with long, fibrous dried and fresh beet leaf remnants that were attached to the beet roots and passed through the first grinder without much size reduction. The decision was made to remove the remaining leaves from roots harvested during Trial #2, thereby reducing the mass from 7.7 tons to 7.07 tons to proceed. Subsequent beets were harvested paying special attention to remove essentially all beet leaves, which eliminated this problem. Ground beets oxidize quickly, likely due to oxidation caused by phenolics, and progress from white in color, to a pinkish-brown color, to a dark brown-black within an hour.

### Steam Injection, Conveying, and Post-Steaming

Due to the variable frequency drive speed range available, a drive-frequency of 20 Hz approximately matched the hammer mill throughput at 2 tons/hr. At this rate, the screw flights were full and formed a plug which steam would visibly penetrate the biomass to a depth of about 2 inches as noticed by observing the discoloration from white/reddish to brown/black. The temperature increase for early trials was from ambient (20-25° C.) to between 60-80° C. depending on exposure of the sample to steam. Average temperatures of bulk mixed samples were about 70° C. Several optimization steps were undertaken to increase the temperature of the beets exiting the steam injection section, which included adjusting the speed of beets fed into the screw feeder, the speed of the screw itself, and the temperature/pressure of the saturated steam entering the screw feeder. Slight gains could be achieved by slowing the screw speed and increasing the steam pressure, however throughput was severely compromised and a high steam temperature risked thermal degradation of the beets and may not be practical at industrial scale. Improving the steam injection process would have been helpful, which could be achieved in practice by increasing the steam injection area by increasing the number of injection ports (inside and outside the shaft) and/or extending the length of the unit. Also, a significant portion of the steam injected into the beets passed through the beets and exited the feeder as lost energy to the atmosphere. Better sealing and insulation could be installed if desired in the future, however, as these modifications would be capital and time intensive, the decision was made to pre- and post-heat the beets entering and leaving the unit as described elsewhere. With an incoming temperature of approximately 35° C. from the preheating process, the outlet temperature of the beets ranged from 75-95° C., averaging 85° C.

As shown in Table 11, the average total solids content for the raw ground beets ranged from 19.8-22.2% for these trials. Table 12 also shows the initial sucrose contents for beets processed during trials 4, 5, and 7, which averaged 12.5% wet-based (60.1% dry-basis) for the raw beets, and 12.0% wet-basis (58.9% dry-basis) after stream injection in the conveyor. These sucrose values are on the low end of the expected range of 60-75% (dry-basis) for sugar beets based on previous work.

Table 12, shows these values for trials 4, 5, and 7, as well as the solids content upon leaving the steam injection conveyor. The solids content for trial 4 increased, which is not expected or explainable based on the fact water is being added, however for trials 5 and 7, the average solids content decreased by approximately 1-1.5 percentage points resulting from steam addition. It is important to note that this is not the final moisture content prior to the start of fermentation. Additional steaming of approximately 30-minutes was required to raise the overall bulk temperature from 85° C. to 100° C. as well as moisture added through enzyme and yeast addition as described later. However, samples from fermentation trial 4 at the time of enzyme addition show the total solids content was still 19%, a total loss of less than 2-percentage points from steam injection.

Table 12 also shows the initial sucrose contents for beets processed during trials 4, 5, and 7, which averaged 12.5% wet-basis (60.1% dry-basis) for the raw beets, and 12.0% wet-basis (58.9% dry-basis) after steam injection in the conveyor. These sucrose values are on the low end of the expected range of 60-75% (dry-basis) for sugar beets based on previous work.

TABLE 18
Solids and Sucrose Contents for Ground and Steamed* Beets

| | Trial # | | |
|---|---|---|---|
| | 4 | 5 | 7 |
| Raw Beets | | | |
| Solids (% wb) | 20.3 ± 0 | 20.7 ± 0.4 | 21.6 ± 0.1 |
| Sucrose (% wb) | 13.3 ± 0.0 | 10.1 ± 0.9 | 14.2 ± 0.4 |
| Sucrose (% db) | 65.7 | 48.8 | 65.9 |
| Steamed Beets* | | | |
| Solids (% wb) | 20.8 ± 0 | 19.6 ± 0.2 | 20.0 ± 0.1 |
| Sucrose (% wb) | 11.3 ± 0.2 | 11.4 ± 0.0 | 13.3 ± 0.5 |
| Sucrose (% db) | 54.5 | 58.4 | 66.5 |

During conveying with the inclined drag-chain conveyor, a small amount of liquid (˜4 L/ton of beets) was lost from the conveyor as steamed beets were moved to the top of the fermenter. This dark black liquid was collected in a drip pan and added back manually periodically using a shovel, although approximately half was ultimately lost.

Once the target temperature of 100° C. was achieved, cooling water was applied to the jacket while mixing in order to rapidly cool the fermenter contents. Cooling needed to be done carefully when cooling water was first applied to as to avoid creating a vacuum condition inside the fermenter and damaging the tank. Overall processing times for a 5-ton batch generally consisted of the following: Washing (2.5 hours), Pre-Steaming (2 hours), Grinding/Milling/Conveying (2.5 hours), Post-Steaming (0.5 hours), Cooling (6 hours).

### Fermentation

As the exact composition of beets for each batch was not known during the trial and the actual wet mass and initial total solids contents varied slightly for batches 4, 5 & 7 from 5.1-5.4 tons/batch, and 20.3-21.6% TS, respectively, enzyme loading was performed on a volumetric loading assumption, and assuming a batch size of 5-tons at 20% TS content. Actual amounts and total solids were measured accurately in the process of the trials and corrected volumetric enzyme loading rates are shown in Table 13. Additionally, although exact compositions were not analyzed, if cellulose, hemicellulose, and pectin fractions are to be consistent and vary in amount only with total solids fractions, total and specific enzyme loading can be calculated (assuming cellulose=5.5%, hemicellulose=4.9%, and pectin=11.7% dry-basis from average previous similar results). It is emphasized that this is an approximate guideline, not exact measurement, but illustrates the range of loadings across batches with this assumption.

TABLE 13
Actual Enzyme Loadings (Trials 4, 5 & 7)

| | Trial # | | |
|---|---|---|---|
| Enzyme | 4 | 5 | 7 |

Volumetric Enzyme Loading

| | | | | |
|---|---|---|---|---|
| Cellulase - CTEC2 | 37.8 | 31 | 37.9 | ml/kg TS |
| Hemicellulase - HTEC2 | 3.8 | 3.1 | 3.8 | ml/kg TS |
| Pectinase - NS22119 | 2.7 | 2.2 | 2.7 | ml/kg TS |

Total Enzyme Activity Loading

| | | | | |
|---|---|---|---|---|
| Cellulase - CTEC2 | 4 | 4 | 4 | FPU/g TS |
| Hemicellulase - HTEC2 | 31 | 30 | 31 | XU/g TS |
| Pectinase - NS22119 | 23 | 22 | 23 | PGU/g TS |

Specific Enzyme Activity Loading

| | | | | |
|---|---|---|---|---|
| Cellulase - CTEC2 | 72 | 71 | 72 | FPU/g Cellulose |
| Hemicellulase - HTEC2 | 624 | 613 | 623 | XU/g Hemicellulose |
| Pectinase - NS22119 | 194 | 190 | 193 | PGU/g Pectin |

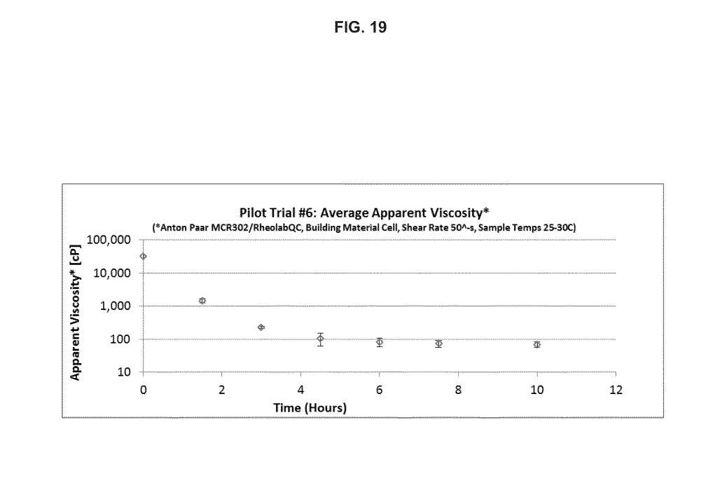

The rotary fermenter worked well for agitation of solid beets as observed from the uniformity of the bed prior to and after addition of enzymes and yeast to the fermenter. The reactor power input for mixing is in the 1-3 kW/m³ reactor volume range using a simple calculation based on the motor HP and total or working volume of the reactor. Amperage data was not monitored however samples were collected every 1.5 hours for the first 10-hours of trial #6 and measured using a rotational rheometer with stirrer and measurement cell designed for testing building materials with particles up to 5 mm-diameter. Results are shown below in FIG. 19 and the contents were well liquefied by 4.5-hours to a viscosity of approximately 100 cp for the conditions shown.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

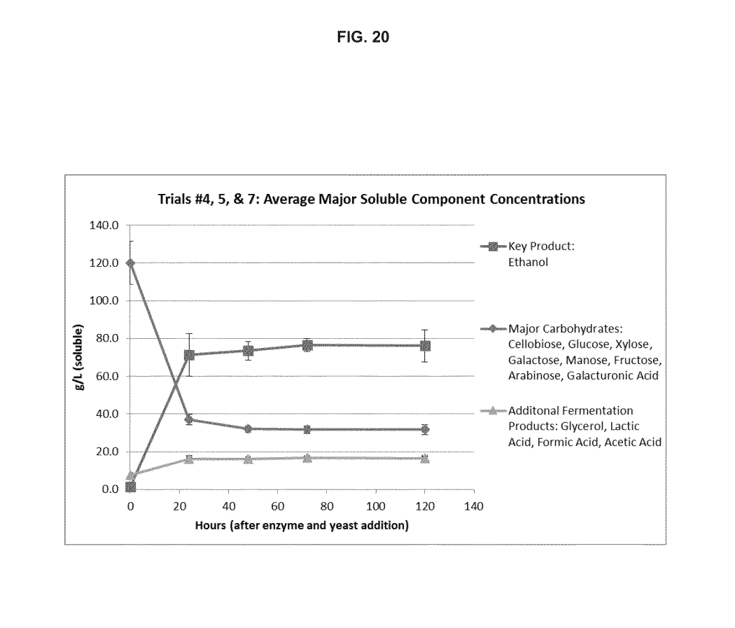

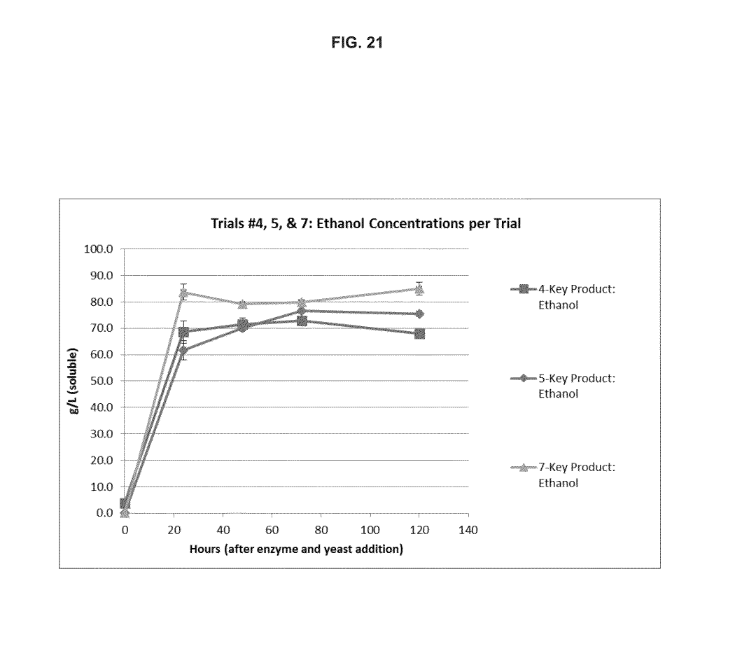

As shown below in FIG. 20 ethanol production was over 90% complete within 24-hours, averaging 71.3±11.2 g/L with a yeast ethanol productivity of 3.0 g/L/hr. Maximum average concentrations were observed on day-3 at 76.5 [symbol] 3.4 g/L. To demonstrate the variability in triplicate ethanol samples analyzed for each trial, individual results for each trial are shown in FIG. 21. Also shown in FIG. 20 are major soluble carbohydrates that are consumed or solubilized. Unhydrolyzed sucrose is not shown in FIG. 20, which is surmised to account for the difference in total carbohydrate expected at time zero, given the ethanol production level and approximate theoretical stoichiometric yield of 1 gram ethanol per 2 grams of carbohydrate.

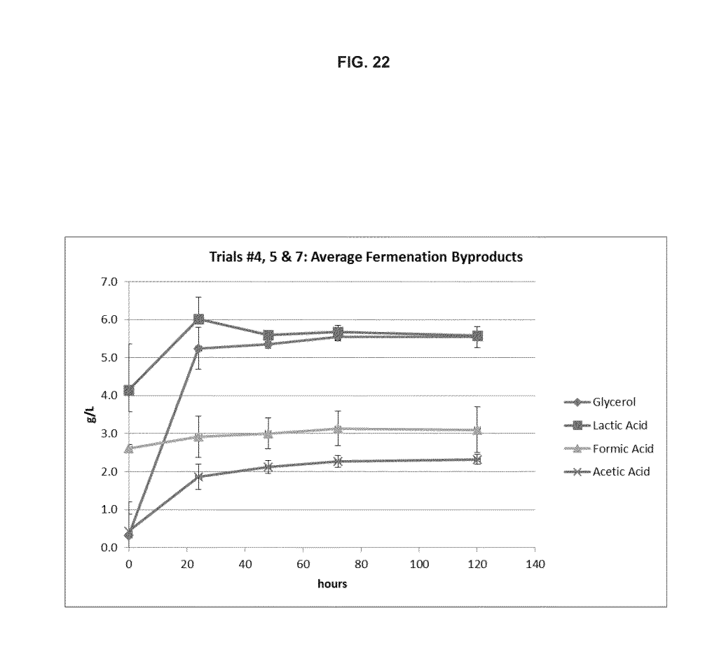

Also shown in FIG. 20, and detailed in FIG. 22, are fermentation byproducts released or produced during fermentation. At the time of yeast and enzyme addition, lactic and formic acid concentrations were approximately 5 g/L and 3 g/L, respectively, and only increased slightly during fermentation. These are indications of inherent microbial activity occurring during the storage and processing stages and areas for control and reduction in the future. Glycerol and acetic acid concentrations, however, increased from near zero to approximately 5 g/L and 2 g/L during the course of the fermentation, with most of the increase occurring in the first 24-hours, and indicate yeast and enzyme coupled activity. Glycerol is a known byproduct of yeast metabolism necessary to some extent for balancing redox potentials and to counter osmotic stress. A value of 5 g/L is not unusual however nitrogen availability and reduced fermentation temperature might be explored to see if this can be lowered. Acetic acid production is likely either from microbial contamination or produced during hydrolysis of the feedstock (primarily hemicellulose and pectin). A level of 2 g/L is below typical yeast inhibitory concentrations around 10 g/L.

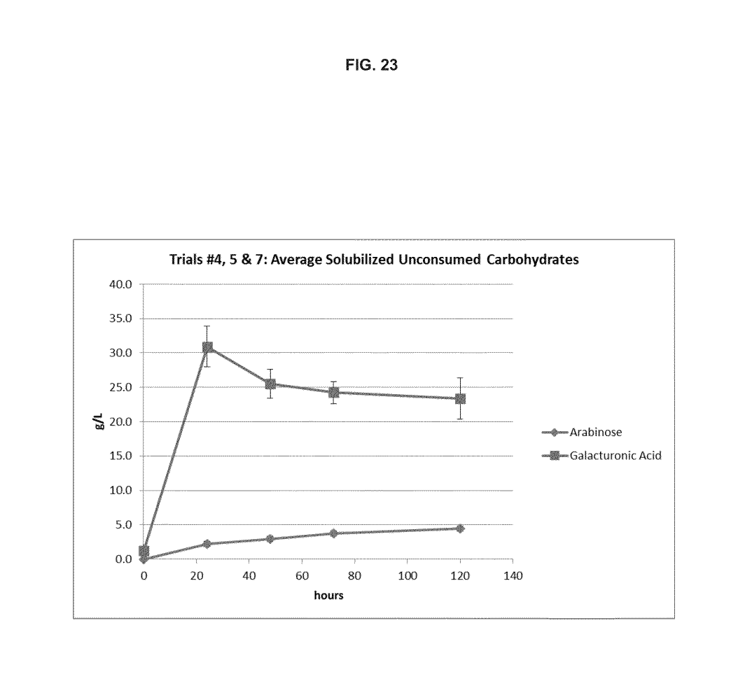

The major carbohydrates left unconsumed are the five carbon sugar arabinose, and the sugar-acid, galacturonic acid, as shown in FIG. 23, neither of which are known to be consumed by wild-type Saccharomyces cerevisiae. Galacturonic acid, the major unconsumed substrate at over 25 g/L, is hydrolyzed mainly in the first 24-hours, while arabinose appears to increase steadily over the course of several days, reaching around 5 g/L final concentration on average. The slight reduction in galacturonic acid could be due to consumption by contaminating organisms although more research would be needed to conclude this.

### Solids Separation and Liquid Transfer to Beer Storage

After 5-days of fermentation, the liquid was transferred to the beer storage tank by draining through the interior racking screen. A thin layer of slurry-like sediment was retained, which was scraped and loaded into a macro-bin by rotating the fermenter in the “unloading” direction prior to washing out any remaining solids for cleaning purposes. The collected solids were weighed and tested for total solids content. For example, Trial #7 contained 1226 pounds of residual solids at 12% solids were collected. The volume of liquid in the beer storage tank was recorded and used to calculate the total final ethanol amount in each batch. Final results for ethanol production and yield are shown in the final mass balance tables shown in Section 3.6 for trials 4, 5 and 7 individually.

### Ethanol Removal

Ethanol removal was conducted in a 250-gallon steam jacketed kettle with additional internal copper heating coil and operated at a working volume of 200-gallons per batch. Approximately 3600 gallons of beer from batches 4, 5 and 7 were processed in this fashion to evaporate ethanol and create a stillage to be fed to anaerobic digestion. Beer and stillage from several batches were combined for processing purposes. As no ethanol or water vapor recovery system was in place, approximately 30% of the volume and mass of the beer was lost to the atmosphere as ethanol and water vapor in creating the stillage product. The time to process one 200-gallon batch of was between 2-3 hours, significantly longer than estimated initially. As such, the first several batches were boiled for several hours, but only reached a final temperature of 98-99° C. before being transferred to stillage storage. This material was subsequently tested and found to contain ˜20 g/L of ethanol, or only about ⅓-¼ of the original removed. This is not unexpected as the energy required to remove ethanol from water increases as the concentration decreases towards the boiling point of pure water. For the last 3 batches of stillage produced, boiling for over 4-hours per batch was performed and a final temperature of 100° C. obtained as measured by the digital temperature probe, however, the final ethanol concentration was tested to still be approximately 10 g/L. The average ethanol concentration for the blended stillage feedstock was therefore in the 15 g/L range.

Since approximately 30% of the total initial beer mass was lost during the boiling process and 700-1000 gallons additional stillage was diverted for fertilizer testing and loss upon transfer, of the initial beer processed, only 1500 gallons were reserved as stillage for destined for anaerobic digester addition. Significant foaming was noticed during the boiling process and should be considered in the design of an industrial distillation process with this substrate.

### Actual Mass Balance and Ethanol Yield Calculations

Mass balance tabulations for trials 4, 5 & 7 are shown in the next section in Table 24, Table 25, and Table 26, respectively. Values estimated from analytical measurements obtained are shown in red text, while values estimated or assumed are shown in black text. A density of 8 lb/gallon for the beer is assumed for all trials. Initial sucrose contents were measured but detailed structural compositions were not at the time of this report. Stoichiometric conversion of hexose to ethanol is assumed to estimate a mass of carbon dioxide lost.

For trial #4, a maximum value of 73 g-Ethanol/L was achieved and therefore approximately 112 gallons of ethanol were produced. Given that 1240 gallons of beer were transferred to the storage tank, an estimated mass balance closure of 123% is observed. Mass of solids remaining in the fermenter were not measured for this batch, but estimated from other batch results. For an initial sucrose value of 133 g/L measured, approximately 100% of theoretical conversion based on initial sucrose and 92% of assumed total hexose conversion is achieved. The fermentation ethanol yield is 0.33 g-Ethanol/g-initial total solids, or a process yield of approximately 20.7 gallons-ethanol/initial wet ton of beets. Approximately 29% of the beer mass was assumed lost as vapor during stillage production.

For trial #5, a maximum value of 77 g-Ethanol/L was achieved and therefore approximately 120 gallons of ethanol were produced. Given that 1200 gallons of beer were transferred to the storage tank, an estimated mass balance closure of 101% is observed. Mass of solids remaining in the fermenter were not measured for this batch, but estimated from other batch results. For an initial sucrose value of 101 g/L measured, well over 100% of theoretical conversion based on initial sucrose and total hexose conversion is achieved, however, based on expected sucrose contents and results from other trials, the initial sucrose measurement is assumed to be lower than the actual amount present. The fermentation ethanol yield is 0.35 g-Ethanol/g-initial total solids, or a process yield of approximately 22.7 gallons-ethanol/initial wet ton of beets. Approximately 29% of the beer mass was assumed lost as vapor during stillage production.

For trial #7, a maximum value of 87 g-Ethanol/L was achieved and therefore approximately 132 gallons of ethanol were produced. Given that 1100 gallons of beer were transferred to the storage tank, an estimated mass balance closure of 99% is observed. Mass of solids remaining in the fermenter was measured to be 1226 lbs (or ˜12% of total mass) at 12% solids content for this batch. For an initial sucrose value of 142 g/L measured, over 100% of theoretical conversion based on initial sucrose and 100% of assumed total hexose conversion is achieved. The fermentation ethanol yield is 0.39 g-Ethanol/g-initial total solids, or a process yield of approximately 25.8 gallons-ethanol/initial wet ton of beets. Approximately 23% of the beer mass was assumed lost as vapor during stillage production.

Overall process yield on a gallon/wet-ton-of-beets basis is highly dependent on the initial solids content for the beets, which for these trials were in the 20-21% solids range.

All publications, patents, and patent applications cited herein are hereby incorporated by reference in their entirety for all purposes.

What is claimed is:

1. A process for preparing ethanol from a first agricultural feedstock comprising a soluble saccharide, which is a fermentable ethanol precursor, and about 20% total solids (TS), said method comprising:
(a) mechanically processing said first agricultural feedstock to prepare a first hydrolysis feedstock comprising a solid feedstock comprising said fermentable ethanol precursor;
(b) submitting said first agricultural feedstock to a heat pretreatment under conditions sufficient to reduce microbial contamination of said agricultural feedstock;
(c) in a hydrolysis vessel, contacting said first hydrolysis feedstock with a hydrolytic enzyme capable of liquefying said solid feedstock under conditions sufficient to liquefy said solid feedstock, forming a first fermentation substrate; and
(d) in a fermentation vessel, contacting said first fermentation substrate with a microorganism capable of converting said fermentation substrate to ethanol under conditions sufficient to convert said fermentable ethanol precursor to a first ethanol fraction.

2. The process according to claim 1, wherein said hydrolysis vessel and said fermentation vessel are different vessels.

3. The process according to claim 1, wherein said hydrolysis vessel and said fermentation vessel are the same vessel.

4. The method according to claim 1, wherein said first agricultural feedstock is selected from a root, a fruit and a vegetable.

5. The method according to claim 4, wherein said first agricultural feedstock is selected from a beet, a melon and potato.

6. The method according to claim 1, wherein said first agricultural feedstock is the root of a sugar beet.

7. The method according to claim 1, wherein said first agricultural feedstock has a high soluble carbohydrate, high pectin and low lignin content.

8. The method according to claim 1, wherein said heat pretreatment is before a member selected from step (a), step (b) and a combination thereof

9. The method according to claim 8, wherein said heating is at a temperature of from about 70° C. to about 130° C. for a time of from about 5 minutes to about 120 minutes.

10. The method according to claim 9, wherein said heating is at a temperature of from about 95° C. to about 100° C. for a time of about 15 minutes to about 20 minutes.

11. The method according to claim 10, wherein said heating is at a temperature of about 70° C. for a time of about 120 minutes.

12. The method according to claim 8, wherein said heating decreases microbial activity in said hydrolysis feedstock.

13. The method according to claim 8, wherein said heating is not accompanied by significant extraction of said fermentable feedstock from said solid feedstock.

14. The method according to claim 8, wherein said heating is not accompanied by addition of a significant amount of water to said hydrolysis feedstock.

15. The method according to claim 8, wherein said heating is accompanied by the addition of no more than about 5% (w/w) water to said hydrolysis feedstock.

16. The method according to claim 1, wherein said fermentation substrate is a soluble saccharide.

17. The method according to claim 16, wherein said fermentation substrate contains a member selected from sucrose, glucose, fructose and a combination thereof.

18. The method according to claim 1, wherein said liquefying reduces mechanical strength of said first agricultural feedstock by from about 50% to about 100%.

19. The method according to claim 1, wherein viscosity of said first hydrolysis feedstock in said hydrolysis vessel is quantified and when said viscosity has reached a predetermined viscosity threshold, said first hydrolysis feedstock is transferred to said fermentation vessel.

20. The method according to claim 19, wherein said predetermined viscosity threshold is from about 500 cp to about 1000 cp.

21. The method according to claim 1, wherein said hydrolytic enzyme hydrolyses a member selected from an oliogsaccharide, a polysaccharide, and a combination thereof.

22. The method according to claim 21, wherein said oligosaccharide is pectin.

23. The method according to claim 1, wherein said hydrolytic enzyme is a member selected from a cellulase, a hemi-cellulase, a pectinase, a β-glucosidase and a combination thereof.

24. The method according to claim 1, wherein said hydrolytic enzyme is a combination of a pectinase and a cellulase.

25. The method according to claim 1, wherein said hydrolysis vessel is maintained at a temperature of from about 25° C. to about 90° C.

26. The method according to claim 1, wherein said process further comprises, introducing into said process a second agricultural feedstock.

27. The method according to claim 26, wherein said second agricultural feedstock is lignocellulosic biomass.

28. The method according to claim 27, wherein said lignocellulosic biomass is selected from leaves, grass, straw and a combination thereof.

29. The method according to claim 28, wherein said lignocellulosic biomass is sugar beet leaf biomass.

30. The method according to claim 26, further comprising:
(f) mechanically processing said second agricultural feedstock to prepare a second hydrolysis feedstock comprising a solid feedstock;
(g) in a second hydrolysis vessel, contacting said second hydrolysis feedstock with a hydrolytic enzyme capable of liquefying said solid feedstock under conditions sufficient to liquefy said solid feedstock, forming a second fermentation substrate; and
(h) in a second fermentation vessel, contacting said second fermentation substrate with a microorganism capable of converting said second fermentation substrate to ethanol under conditions sufficient to convert said fermentable ethanol precursor to a second ethanol fraction,
h).

31. The method according to any preceding claim, further comprising:
(d) removing at least a portion of said ethanol from said fermentation liquid, such that stillage is created.

32. The method according to claim 31, further comprising, (e) contacting said stillage with a microorganism capable of converting said stillage to ethanol under conditions sufficient to convert said stillage to a third ethanol fraction.

33. The method according to claim 1, wherein said ethanol is removed from said fermentation vessel.

34. The method according to claim 33, wherein said ethanol is removed by a method selected from distillation and membrane separation.

35. The method according to claim 27, wherein said ethanol removed from said fermentation vessel is added to a member selected from said first fermentation vessel, said second fermentation vessel and a combination thereof.

36. The method according to claim 1, wherein said microorganism is selected from bacteria, yeast or a combination thereof.

37. The method according to claim 30, further comprising, (i) submitting said second agricultural feedstock to a heat pretreatment under conditions sufficient to reduce microbial contamination of said second agricultural feedstock, wherein said heat pretreatment is before a member selected from step (f), step (g) and a combination thereof.
